(19) United States
(12) Patent Application Publication
(10) Pub. No.: US 2023/0381413 A1
(43) Pub. Date: Nov. 30, 2023

(54) TIME-SEGMENTED BASAL RATE ADAPTATION PROCESS

(71) Applicant: Beta Bionics, Inc., Concord, MA (US)

(72) Inventors: Michael J. Rosinko, Las Vegas, NV (US); Firas H. El-Khatib, Allston, MA (US); Edward R. Damiano, Acton, MA (US)

(21) Appl. No.: 18/322,678

(22) Filed: May 24, 2023

Related U.S. Application Data
(60) Provisional application No. 63/365,594, filed on May 31, 2022.

(51) Int. Cl. A61M 5/172; G16H 20/17; A61B 5/145

(52) U.S. Cl. CPC A61M 5/1723; G16H 20/17; A61B 5/14532

(57) ABSTRACT

A glucose level control system may be configured to adapt a time-segmented basal dose. The glucose level control system may determine the first basal dose based at least in part on a user-specified basal dose and/or total daily dose value. The first basal dose may also be based on a basal dose received from the memory of the glucose level control system or from a remote storage device. The first basal dose may be administered during a first time period. The glucose level control system may adapt the first basal dose to a second basal dose for a second therapy period. The glucose level control system may adapt the second basal dose based on a difference between a predicted and measured glucose level.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### TECHNICAL FIELD

The present disclosure relates to ambulatory medical devices, such as glucose level control systems, that provide therapy to a subject.

### BACKGROUND

Sustained delivery, pump driven medicament injection devices generally include a delivery cannula mounted in a subcutaneous manner through the skin of the patient at an infusion site. The pump draws medicine from a reservoir and delivers it to the patient via the cannula. The injection device typically includes a channel that transmits a medicament from an inlet port to the delivery cannula which results in delivery to the subcutaneous tissue layer where the delivery cannula terminates. Some infusion devices are configured to deliver one medicament to a patient while others are configured to deliver multiple medicaments to a patient.

Some pump driven medicament devices use a closed loop control system to control the amount of medicament supplied to a subject. The closed loop control system may determine the amount of medicament to supply based on one or more sensor readings obtained from one or more sensors configured to measure one or more physiological characteristics of the subject.

### SUMMARY

The systems, methods, and devices of this disclosure each have several innovative aspects, no single one of which is solely responsible for all the desirable attributes disclosed herein. Details of one or more implementations of the subject matter described in this specification are set forth in the accompanying drawings and the description below.

Certain embodiments of the present disclosure relate to a glucose level control system configured to adapt a basal dose of medicament administered to a subject over a first time period. The glucose level control system including: a medicament delivery interface configured to operatively connect to a medicament pump for infusing the medicament into the subject; a memory configured to store specific computer-executable instructions; and a hardware processor in communication with the memory and configured to execute the specific computer-executable instructions. The computer-executable instructions may be configured to at least: obtain, via user interaction with a basal dose entry user interface, an indication of a first basal dose associated with administering the medicament to the subject over the first time period, wherein the first time period is on the order of at least a day; configure the medicament delivery interface to administer the medicament using at least in part the first basal dose over the first time period; determine a predicted glucose level of the subject over the first time period based at least in part on a medicament on board value over the first time period; determine a measured glucose level of the subject during the first time period; determine a difference between the predicted glucose level of the subject and the measured glucose level; and adapt the basal dose of the medicament by modifying the first basal dose to a second basal dose associated with administering the medicament to the subject over a second time period, wherein the second basal dose is based at least in part on the difference between the predicted glucose level of the subject and the measured glucose level.

The system of the preceding paragraph can include any combination or sub-combination of the following features: wherein the hardware processor is further configured to execute the specific computer-executable instructions to at least select a time segment from the plurality of time segments where at least one of a plurality of basal evaluation criteria is satisfied, wherein the plurality of basal evaluation criteria includes: a medicament on board attributable to non-basal medicament delivery is less than or equal to a medicament delivery threshold; a medicament bolus beyond a minimum bolus threshold to compensate for food intake occurred at least 3 hours prior to a beginning of the time segment; the medicament on board attributable to a food-intake bolus is less than or equal to 2 units; a medicament bolus beyond the minimum bolus threshold to compensate for a glucose excursion occurred at least 3 hours prior to the beginning of the time segment; the medicament on board attributable to a correction bolus is less than or equal to 2 units; a glucose level of the subject is within a target range; it is determined that the subject is not exercising; it is determined that the subject is not experiencing a temporary illness that affects the glucose level of the subject; and the medicament on board attributable to food intake and to the correction bolus is less than or equal to 3 units. Wherein the measured glucose level corresponds to the time segment where the at least one of the plurality of basal evaluation criteria is satisfied.

Certain embodiments of the present disclosure relate to a glucose level control system configured to adapt a basal dose of medicament administered to a subject. The glucose level control system including: a medicament delivery interface configured to operatively connect to a medicament pump for infusing the medicament into the subject; a memory configured to store specific computer-executable instructions; and a hardware processor in communication with the memory. The hardware processer configured to execute the specific computer-executable instructions to at least: obtain a first basal dose associated with administering the medicament to the subject during at least a portion of a measurement time period corresponding to a first therapy period, wherein the measurement time period is on the order of hours to days; configure the medicament delivery interface to administer the medicament using at least the first basal dose over the measurement time period; determine a time segment during the measurement time period where at least one of a plurality of basal evaluation criteria is satisfied, wherein the plurality of basal evaluation criteria includes: a medicament on board attributable to non-basal medicament delivery is less than or equal to a medicament delivery threshold; a medicament bolus beyond a minimum bolus threshold to compensate for food intake occurred at least 3 hours prior to a beginning of the time segment; the medicament on board attributable to a food-intake bolus is less than or equal to 2 units; a medicament bolus beyond the minimum bolus threshold to compensate for a glucose excursion occurred at least 3 hours prior to the beginning of the time segment; the medicament on board attributable to a correction bolus is less than or equal to 2 units; a glucose level of the subject is within a target range; it is determined that the subject is not exercising; it is determined that the subject is not experiencing a temporary illness that affects the glucose level of the subject; and the medicament on board attributable to food intake and to the correction bolus is less than or equal to 3 units; receive time-varying glucose level data of the subject during the time segment; determine whether the time-varying glucose level data satisfies one or more basal dose adjustment criteria; and responsive to determining that the time-varying glucose level data satisfies the one or more basal dose adjustment criteria, adjust the basal dose from the first basal dose to a second basal dose, wherein the second basal dose is associated with administering the medicament to the subject over a second therapy period.

The system of the preceding paragraph can include any combination or sub-combination of the following features: wherein the one or more basal dose adjustment criteria includes: a determination that a percentage of time that the time-varying glucose level data of the subject is within the target range does not satisfy a threshold percentage; a determination that the glucose level of the subject is outside of the target range at a conclusion of the time segment; a determination that the glucose level of the subject is outside of the target range for at least a portion of the time segment; a determination that the glucose level of the subject differs from the target range by more than a threshold difference for at least the portion of the time segment; a determination that a slope of the time-varying glucose level data of the subject satisfies a slope threshold; or a determination that a slope of a trendline fit to the time-varying glucose level data of the subject satisfies a trendline slope threshold.

Certain embodiments of the present disclosure relate to a glucose level control system configured to adapt a prediction model used, at least in part, to determine medicament doses to administer to a subject. The glucose level control system including: a medicament delivery interface configured to operatively connect to a medicament pump for infusing the medicament doses into the subject; a memory configured to store specific computer-executable instructions; and a hardware processor in communication with the memory. The hardware processor configured to execute the specific computer-executable instructions to at least: cause a medicament dose to be administered to the subject, wherein the medicament dose is determined by a control algorithm configured to generate a dose control signal based at least in part on a glucose level prediction determined by the prediction model; determine a predicted glucose level of the subject over a first time period using the prediction model and based at least in part on a medicament on board value; determine a measured glucose level of the subject during the first time period; determine a difference between the measured glucose level and the predicted glucose level; and responsive to determining that the difference between the measured glucose level and the predicted glucose level satisfies a threshold difference, adapt the prediction model, wherein adapting the prediction model causes a change in the medicament doses determined by the control algorithm.

The system of the preceding paragraph can include any combination or sub-combination of the following features: wherein determining that the difference between the measured glucose level and the predicted glucose level satisfies the threshold difference includes one or more of the following: determining that a difference between a minimum number or percentage of the plurality of measured glucose levels and corresponding plurality of predicted glucose levels satisfies the threshold difference; determining that an average difference between the plurality of measured glucose levels and the corresponding plurality of predicted glucose levels satisfies the threshold difference; or determining that a trend in a difference between the plurality of measured glucose levels and the corresponding plurality of predicted glucose levels satisfies the threshold difference.

Certain embodiments of the present disclosure relate to a glucose level control system configured to adapt a total daily dose value corresponding to a total daily dose of a medicament administered to a subject. The glucose level control system including: a medicament delivery interface configured to operatively connect to a medicament pump for infusing the medicament into the subject; a memory configured to store specific computer-executable instructions; and a hardware processor in communication with the memory. The hardware processor configured to execute the specific computer-executable instructions to at least: obtain a first total daily dose value corresponding to the total daily dose of medicament administered to the subject; cause medicament therapy to be administered to the subject during a first therapy period based at least in part on the first total daily dose value, wherein the first therapy period is on the order of at least a day, and wherein the medicament therapy is determined by a control algorithm configured to generate a dose control signal based at least in part on the first total daily dose value and a glucose level of the subject; obtain therapy data corresponding to the medicament therapy administered during the first therapy period; determine, based at least in part on the therapy data, that at least one of a plurality of therapy data evaluation criteria is satisfied; and responsive to determining that at least one of the plurality of therapy data evaluation criteria is satisfied, adapt the total daily dose value by replacing the first total daily dose value with a second total daily dose value associated with administering the medicament to the subject over a second therapy period, wherein the control algorithm is further configured to generate a dose control signal based at least in part on the second total daily dose value.

The system of the preceding paragraph can include any combination or sub-combination of the following features: wherein the plurality of therapy data evaluation criteria includes: a determination that the first total daily dose value of the subject does not match, within a threshold degree, an administered daily medicament dose value during the first therapy period; a determination that a difference between the first total daily dose value and the administered daily medicament dose value exceeds a difference threshold for a minimum number of days during the first therapy period; a determination that a trend of the difference between the first total daily dose value and the administered daily medicament dose value exceeds a trend threshold; a determination that an average difference between the first total daily dose value and the administered daily medicament dose value during the first therapy period exceeds an average difference threshold; a determination that a threshold amount of time has passed since the total daily dose was adapted; and a determination that a particular time period has been reached.

### BRIEF DESCRIPTION OF THE DRAWINGS

Throughout the drawings, reference numbers are re-used to indicate correspondence between referenced elements. The drawings are provided to illustrate certain aspects of the subject matter described herein and not to limit the scope thereof.

FIG. 1A illustrates a first example of an ambulatory medicament device environment for controlling a glucose level of a subject via an ambulatory medicament pump.

FIG. 1B illustrates a second example of an ambulatory medicament device environment for controlling a glucose level of a subject via an ambulatory medicament pump.

FIG. 1C illustrates a third example of an ambulatory medicament device environment for controlling a glucose level of a subject via an ambulatory medicament pump.

FIG. 2A illustrates a block diagram of a first example of a glucose level control system.

FIG. 2B illustrates a block diagram of a second example of a glucose level control system.

FIG. 2C illustrates a block diagram of a third example of a glucose level control system.

FIG. 2D illustrates a block diagram of a fourth example of a glucose level control system.

FIG. 3 illustrates a block diagram of an example glucose level control system that includes an electronic communications interface.

FIG. 4A illustrates a block diagram of an example of elements of a glucose level control system operating in an online mode.

FIG. 4B illustrates a block diagram of an example of elements of a glucose level control system operating in an offline mode.

FIG. 5A illustrates a block diagram of an example glucose level control system environment that includes a glucose level control system in accordance with certain embodiments.

FIG. 5B illustrates a block diagram of a second example glucose level control system environment in accordance with certain embodiments.

FIG. 5C illustrates a block diagram of a third example glucose level control system environment in accordance with certain embodiments.

FIG. 6 illustrates a block diagram of an example controller in accordance with certain embodiments.

FIG. 7 presents a flowchart of an example basal dose adaptation process in accordance with certain embodiments.

FIG. 8 presents a flowchart of an example time-segmented dose adaptation process in accordance with certain embodiments.

FIG. 9 presents a flowchart of an example predication model adaptation process in accordance with certain embodiments.

FIG. 10 presents a flowchart of an example total daily dose adaptation process in accordance with certain embodiments.

### DETAILED DESCRIPTION OF CERTAIN EMBODIMENTS

In some cases, a subject's medicament needs may periodically change. For example, keeping a subject's glucose level within a desired range may require more or less insulin during particular times of the day. For instance, a subject may need more insulin in the mornings than in the evenings. If a subject's medicament doses (e.g., basal doses) do not account for the subject's changing medicament needs, the subject's glucose level may rise or fall to unhealthy levels.

Advantageously, the glucose level control system described herein may be configured to administer a time-segmented basal dose. The time-segmented basal dose may be based on subject's past therapy data and may anticipate periodic changes in the subject's medicament needs. For example, if the subject's glucose level tends to rise in the evenings, a basal dose that corresponds to the evenings can be increased accordingly. Thus, the glucose level control system may prevent the subject from experiencing hypoglycemic and hyperglycemic events. Additionally, the glucose level control system may also adapt the time-segmented basal dose based on the subject's glucose level history, therapy data, total daily dose of medicament, and/or medicament on board value. The adapted time-segmented basal dose may reduce fluctuations in the subject's glucose level and may increase the amount of time that the subject's glucose level is within a target range. The glucose level control system may perform the time-segmented basal dose adaptation process multiple times.

The time-segmented basal dose adaptation process may use a control algorithm and/or a prediction model. In some cases, one or more parameters of the control algorithm are set based at least partly on a user-inputted basal dose or total daily dose value. The glucose level control system may adapt one or more parameters over time based at least in part on user-inputted values, the subject's past therapy history, on board medicament values, total daily dose values, and/or glucose levels. In some cases, the glucose level control system may adapt the parameters to adjust the medicament deliveries to reduce the error between a subject's predicted glucose level and measured glucose level.

In some non-limiting examples, the glucose level control system may perform a prediction model adaptation process. In some cases, the prediction model adaptation process may change the weight and/or relationships of one or more parameters of the predication model. Adapting the prediction model may cause the glucose level control system to adapt the subject's medicament therapy more effectively.

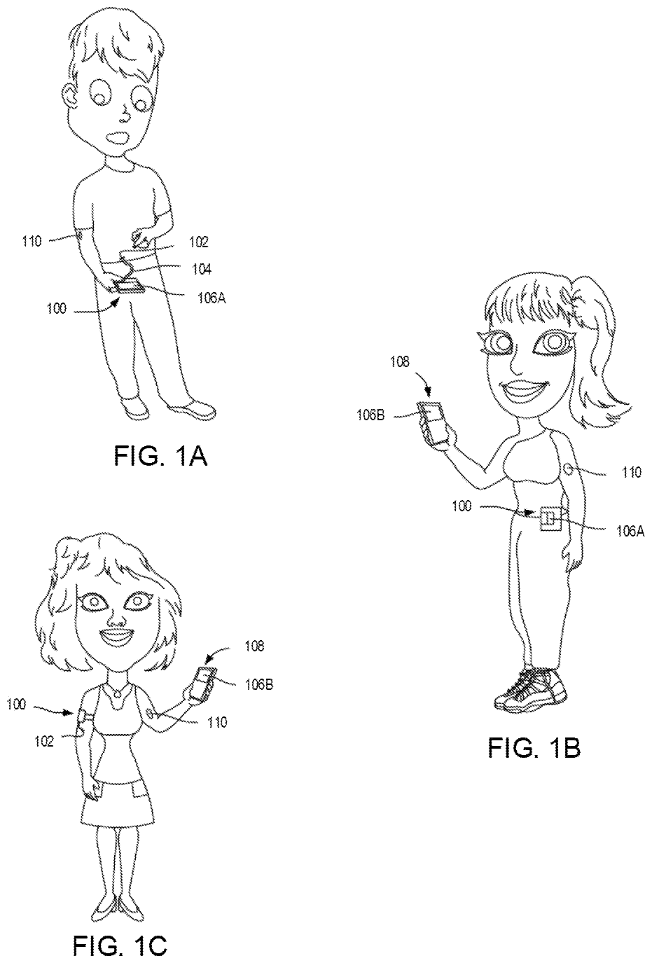

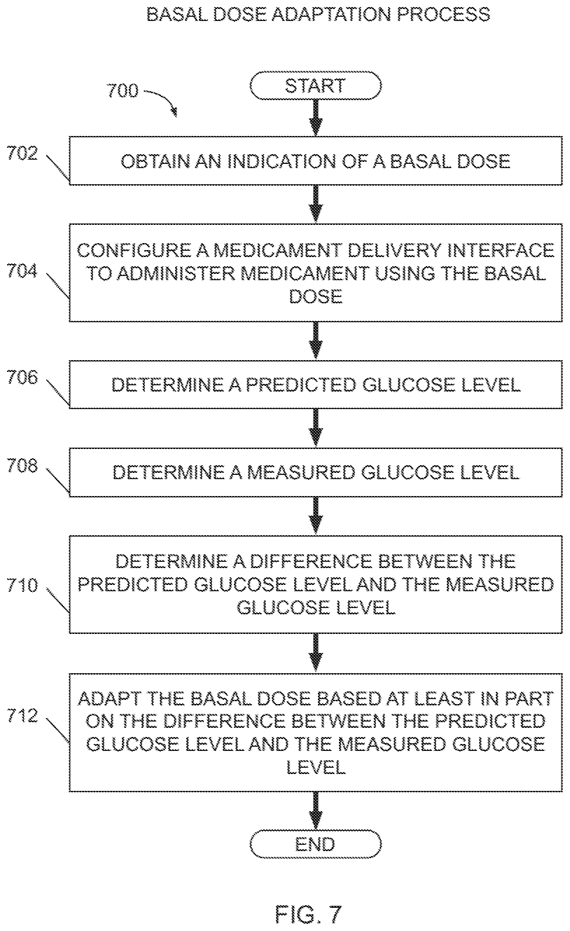

Detailed descriptions and examples of systems and methods according to one or more embodiments may be found in the section entitled Adaptive Glucose Level Control System, basal dose adaptation process, time-segmented basal dose adaptation process, prediction model adaptation process, and total daily dose adaptation process as well as in the section entitled Example Embodiments, and also in FIGS. 7-10 herein. Furthermore, embodiments disclosed herein may be implemented by components and functionality for time-segmented basal dose adaptation process that may be configured and/or incorporated into glucose level control systems, such as those described herein in FIGS. 1A-1C, 2A-2D, 3, 4A, 4B, 5, and 6.

### Infusion System Overview

Some embodiments described herein pertain to medicament infusion systems for one or more medicaments and the components of such systems (e.g., infusion pumps, medicament cartridges, cartridge connectors, lumen assemblies, infusion connectors, infusion sets, etc.). Some embodiments pertain to methods of manufacturing infusion systems and components thereof. Some embodiments pertain to methods of using any of the foregoing systems or components for infusing one or more medicaments (e.g., pharmaceutical, hormone, etc.) to a subject or patient. As an exemplary illustration, an infusion system may include an infusion pump, which can include one or more medicament cartridges or can have an integrated reservoir of medicament. The infusion pump may also be referred to as a medicament pump. An infusion system may include medicament cartridges and cartridge connectors, but not a pump. An infusion system may include cartridge connectors and an infusion pump, but not medicament cartridges. An infusion system may include infusion connectors, a lumen assembly, cartridge connectors, an infusion pump, but not medicament cartridges or an infusion set.

A glucose level control system can operate in conjunction with an infusion system to infuse one or more medicaments, including at least one glucose level control agent, into a subject. The glucose level control agent may include insulin, an insulin analog, a counter-regulatory agent, or any other pharmaceutical or hormone that may affect a subject's glucose level or glucose sensitivity (e.g., metformin, prandin, canagliflozin, etc.).

Any feature, structure, component, material, step, or method that is described and/or illustrated in any embodiment in this specification can be used with or instead of any feature, structure, component, material, step, or method that is described and/or illustrated in any other embodiment in this specification. Additionally, any feature, structure, component, material, step, or method that is described and/or illustrated in one embodiment may be absent from another embodiment.

### Glucose Level Control System Overview

Glucose level control systems are used to control a glucose level (such as, but not necessarily, a blood glucose level) in a subject or patient. In some aspects, the glucose level may comprise a blood glucose level, or a glucose level in other parts or fluids of the subject's body. In some examples, the glucose level may comprise a physiological glucose level of the subject that can be a concentration of glucose in the subject's blood or an interstitial fluid in a part of the subject's body (e.g., expressed in milligram per deciliter (mg/dL or sometimes mg/dl)).

Glucose level control systems (GLCSes), which can also be referred to herein as glucose control systems or glucose level systems, can include a controller configured to generate dose control signals for one or more glucose control agents that can be infused into the subject. Glucose control agents can include regulatory agents that tend to decrease a subject's glucose level, such as insulin and insulin analogs, and counter-regulatory agents that tend to increase a subject's glucose level, such as glucagon or dextrose. In some cases, glucose control agents may include alternative hormones or pharmaceuticals that can affect a subject's glucose level or glucose sensitivity, such as metformin, prandin, canagliflozin, etc. A glucose level control system configured to be used with two or more glucose control agents can generate a dose control signal for each of the agents, or medicaments. In some embodiments, a glucose level control system can generate a dose control signal for an agent even though the agent may not be available for dosing via a medicament pump connected or operatively connected to the subject. Further, in some embodiments, a glucose level control system can generate an indication of a recommended dose of an agent or medicament even though the glucose level control system may not be in communication with or in control of a medicament delivery device (e.g., a medicament pump or medicament pen).

Glucose control agents can be delivered to a subject via subcutaneous injection, via intravenous injection, or via another suitable delivery method. In the case of glucose level control therapy via a medicament pump or an ambulatory medicament pump, subcutaneous injection is most common. An ambulatory medicament pump (AMP) is a type of ambulatory medical device, which is sometimes referred to herein as an ambulatory device, an ambulatory medicament device, a mobile ambulatory device, or an AMD. Ambulatory medical devices include ambulatory medicament pumps and other devices configured to be carried by a subject and to deliver therapy to the subject.

In some embodiments, a glucose level control system can implement one or more algorithms relating to medicament control or glucose level control as discussed herein to provide medicament or glucose level control therapy. In some cases, the glucose level control system may operate, at least in part, without being connected to an ambulatory medicament device. For example, the glucose level control system can provide instructions or output a recommendation of a dose of medicament that directs a user to administer the medicament to provide glucose level control therapy. In some implementations, the user may use, for example, a medicament pen to manually or self-administer the medicament according to the glucose level control system's recommended dose outputs. In some implementations, the user may provide inputs, such as glucose level readings to the glucose level control system. The glucose level control system may generate medicament dose recommendations based at least in part on the user inputs. The user inputs to the glucose level control system may be combined with inputs from other systems or devices, such as sensors as discussed herein. In some implementations, the glucose level control system can provide glucose level control therapy based on user inputs without other system or device inputs.

In some embodiments, the glucose level control system includes a memory that stores specific computer-executable instructions for generating a dose recommendation and/or a dose control signal. The dose recommendation and/or the dose control signal can assist with glucose level control of a subject via medicament therapy. The dose recommendation or dose output of the glucose level control system can direct a user to administer medicament to provide medicament therapy for glucose level control, including manual administration of medicament doses. In additional embodiments, the glucose level control system may include a medicament delivery interface that can interact with a medicament delivery device that delivers at least a portion of the medicament therapy. In further embodiments, the glucose level control system may include a sensor interface that can interact with a sensor configured to generate a glucose level signal corresponding to a glucose level of a subject. The glucose level control system can generate the dose recommendation and/or the dose control signal based at least in part on the glucose level signal. In certain embodiments, the dose recommendation and/or the dose control signal can additionally be based at least in part on values of one or more control parameters. Control parameters can include subject-specific parameters, delivery device-specific parameters, glucose sensor-specific parameters, demographic parameters, physiological parameters, other parameters that can affect the glucose level of the subject, or any combination of one or more of the foregoing.

In some examples, the ambulatory medical device may be an electrical stimulation device, and therapy delivery may include providing electrical stimulation to a subject. An example of an electrical stimulation device is a cardiac pacemaker. A cardiac pacemaker generates electrical stimulation of the cardiac muscle to control heart rhythms. Another example of an electrical stimulation device is a deep brain stimulator to treat Parkinson's disease or movement disorders.

### Example Ambulatory Medicament Device Environment

An ambulatory medicament device (AMD) can be a glucose level control system. The glucose level control system can operate can provide glucose level control via an ambulatory medicament pump connected to a subject. FIGS. 1A-1C illustrate a number of different example configurations for connecting an ambulatory medicament pump to a subject.

FIG. 1A illustrates a first example of an ambulatory medicament device environment for controlling a glucose level of a subject via an ambulatory medicament pump 100. In FIG. 1A, the ambulatory medicament pump 100 is connected to an infusion site 102 using an infusion set 104. The ambulatory medicament pump 100 may have integrated user interface or pump controls 106A that permit a user to view pump data and/or change therapy settings via user interaction with the pump controls 106A, such as via user interface elements. An analyte sensor 110, such as a glucose level sensor or a glucose sensor, generates a glucose level signal that that may be received by a glucose level control system, which may be included as part of the ambulatory medicament pump 100 or may be part of a separate device. The analyte sensor 110 may be a bio sensor. In some variants, the analyte sensor 110 can include an insulin level sensor that can generate an insulin level signal that can be received by the glucose level control system. In some variants, the analyte senor 110 can include a glucose level sensor and/or an insulin level sensor. In some variants, the analyte senor 110 may include a continuous glucose monitor (CGM).

FIG. 1B illustrates a second example of an ambulatory medicament device environment for controlling a glucose level of a subject via an ambulatory medicament pump 100. In FIG. 1B, the ambulatory medicament pump 100 communicates with an external electronic device 108 (such as, for example, a smartphone, tablet, smartwatch, smartglasses, etc.) via a wireless data connection. Like the ambulatory medicament pump ambulatory medicament pump 100, the external electronic device 108 may include pump controls 106B that permit a user to view pump data and/or change therapy settings via user interaction with the pump controls 106B. The pump data may be viewed on a display of the external electronic device 108. Further, at least some of the pump controls 106A can be manipulated via user interaction with user interface elements (e.g., the pump controls 106B) of the external electronic device 108. The analyte sensor 110 (e.g., a glucose level sensor) can also communicate with the ambulatory medicament pump 100 via a wired or wireless data connection. Example user interfaces that can be implemented by one or more of the external electronic devices 108, the ambulatory medicament pump 100, a remote electronic device, and/or other electronic devices are shown and described in International or PCT Publication Nos. WO 2021/067767 and WO 2021/011699, the entire contents of which are hereby incorporated by reference herein and made a part of this specification.

FIG. 1C illustrates a third example of an ambulatory medicament device environment for controlling a glucose level of a subject via an ambulatory medicament pump 100. In FIG. 1C, the ambulatory medicament pump 100 includes an integrated cannula that inserts into the infusion site 102 without a separate infusion set, such as with a patch pump. At least some of the pump controls 106B can be manipulated via user interaction with user interface elements of an external electronic device 108. In some instances, pump controls can be manipulated via user interaction with user interface elements generated by a remote computing environment (not shown), such as, for example, a cloud computing service, that connects to the ambulatory medicament pump 100 via a direct or indirect electronic data connection.

FIGS. 1A-1C illustrate certain non-limiting use cases. It should be understood that other use cases are possible. For example, a glucose level control system of the ambulatory medicament pump 100 may implemented separately from the ambulatory medicament pump 100 itself. In some such cases, the glucose level control system may control the ambulatory medicament pump 100 via an interface, which may be wired or wireless. In some other cases, the glucose level control system may communicate with alternative devices that may be used to facilitate care of the subject. For example, the glucose level control system may communicate with an injection pen or smartpen that a user may use to provide therapy to a subject. As another example, the glucose level control system may communicate with a patch pump that may provide therapy to the subject.

A glucose level control system, which may be part of ambulatory medicament pump 100 or implemented as a separate system that may be capable of controlling the ambulatory medicament pump 100, may include a user interface configured to provide one or more of therapy information, glucose level information, and/or therapy control elements capable of changing therapy settings via user interaction with user interface controls. For example, the user can provide an indication of an amount of a manual bolus of medicament. The indication may be provided via a user interface of the glucose level control system or from an electronic device remote from the glucose level control system or the ambulatory medicament pump 100. The user interface can be implemented via an electronic device that includes a display and one or more buttons, switches, dials, capacitive touch interfaces, touchscreen interfaces, or other user interface elements. In some embodiments, at least a portion of the user interface is integrated with an ambulatory medicament pump 100 that can be tethered to a body of a subject via an infusion set configured to facilitate subcutaneous injection of one or more glucose level control agents. In certain embodiments, at least a portion of the user interface is implemented via an electronic device separate from the ambulatory medicament pump 100, such as a smartphone.

### Example Glucose Level Control System Configurations

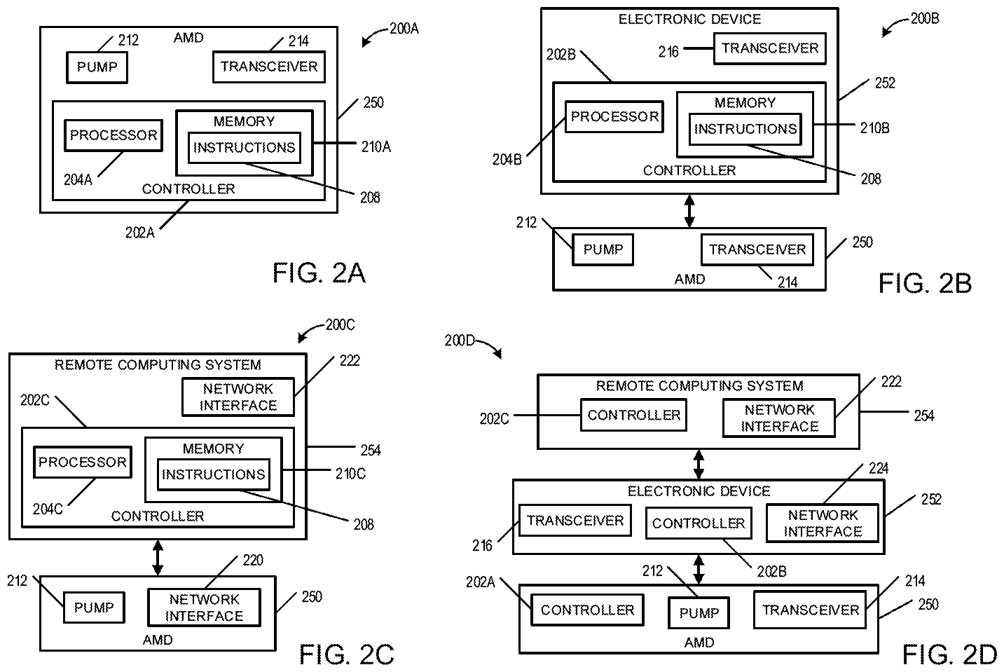

A glucose level control system may be implemented using a number of different configurations that may include a number of different integrated or separate systems. FIGS. 2A-2D illustrate block diagrams of some non-limiting example configurations of a glucose level control system.

FIG. 2A illustrates a block diagram of a first example of a glucose level control system 200A. As illustrated in FIGS. 2A, the glucose level control system 200A can be an ambulatory medicament device 250. The ambulatory medicament device 250 may include a controller 202A. The controller 202A may include an electronic processor 204A and a memory 210A. In some embodiments, the controller 202A may be a separate hardware processor from the electronic processor 204A. Alternatively, the controller 202A may be implemented as software that is executed by the electronic processor 204A.

The memory 210A may be a non-volatile memory, such as flash, a solid-state drive, a hard drive, or any other type of memory that may store instructions 208 executable by the electronic processor 204A. The ambulatory medicament device 250 may further include a medicament pump 212. The medicament pump 212 may include any type of medicament pump that can administer medicament, such as insulin or an insulin analog, to a subject. In some embodiments, the medicament pump 212 may include one or more of the embodiments described with respect to the ambulatory medicament pump 100. When the instructions 208 stored in the memory 210A are executed by the electronic processor 204A, the controller 202A can implement at least a portion of a control algorithm that generates dose control signals for one or more glucose level control agents (e.g., insulin or insulin analogs, or counter-regulatory agents, etc.) based at least in part on time-varying glucose level data corresponding to glucose levels of the subject and/or one or more control parameters. The dose control signals, when delivered to the medicament pump 212, may result in dosing operations to dose medicament that can be used to control the glucose level of a subject.

In some implementations, the ambulatory medicament device 250 may include a transceiver 214. The transceiver 214 may include any type of transceiver that permits or enables wireless communication with another electronic device, such as a router, a smartphone, a laptop, a tablet, a charging station, or any other electronic device that can be configured to communicate with the ambulatory medicament device 250. Using the transceiver 214, the ambulatory medicament device 250 may transmit glucose level data, therapy data (e.g., timing or frequency of medicament delivery), or any other information collected by the ambulatory medicament device 250 to another electronic device.

FIG. 2B illustrates a block diagram of a second example of a glucose level control system 200B. As illustrated in FIG. 2B, in some cases, the glucose level control system 200B can be divided into multiple systems, an ambulatory medicament device 250 and an electronic device 252. The ambulatory medicament device 250 may at least partially be controlled via execution of instructions 208 by the electronic processor 204B of the electronic device 252, which may be separate from an electronic processor 204A of the ambulatory medicament device 250. The electronic device 252 can include a transceiver 216 capable of establishing a wireless connection to the ambulatory medicament device 250 via the transceiver 214 of the ambulatory medicament device 250.

The controller 202B can implement at least a portion of a control algorithm via execution of instructions 208 stored in the memory 210B. When the instructions 208 stored in the memory 210B are executed by the electronic processor 204B, the controller 202B can implement at least a portion of a control algorithm that generates dose control signals for one or more glucose level control agents based at least in part on time-varying glucose level data corresponding to glucose levels of the subject and one or more control parameters. The dose control signals, when delivered to the medicament pump 212, may result in dosing operations that control the glucose level of a subject. The medicament pump 212 may be controlled by at least one pump controller. The pump controller may receive the dose control signals and control the operation of the medicament pump 212 based on the received dose control signals. In some embodiments, the pump controller may be integrated with the medicament pump 212. In various implementations, the controller may be included in the ambulatory medicament device 250, in an electronic device 252 or a remote computing system 254, that are connected to the ambulatory medicament device 250 via wired or wireless communication links. In some embodiments, the dose control signals are transmitted from the transceiver 216 of the electronic device 252 to the transceiver 214 of the ambulatory medicament device 250 over a wired or wireless data connection. The wireless connection may be a short-range wireless connection, such as a Bluetooth® connection, a near-field wireless connection, or any other type of wireless connection. The ambulatory medicament device 250 may receive the dose control signals over the wireless connection and may provide the dose control signals to the medicament pump 212 to administer medicament to a subject.

In some cases, the electronic device 252 may be a remote system. FIG. 2C illustrates a block diagram of a third example of a glucose level control system 200C that can communicate with a remote computing system 254, such as a cloud or network computing system accessible via a network, such as the Internet. The ambulatory medicament device 250 can be at least partially controlled by instructions 208 executed by an electronic processor 204C of a remote computing system 254. When the instructions 208 stored in a memory 210C of the remote computing system 254 are executed by the electronic processor 204C of the remote computing system 254, the controller 202C can implement at least a portion of a control algorithm that generates dose control signals for one or more glucose level control agents based on time-varying glucose level data corresponding to glucose levels of the subject and one or more control parameters. The dose control signals, when delivered to the medicament pump 212, may result in dosing operations that control the glucose level of a subject. In some embodiments, the dose control signals are transmitted from a network interface 222 of the remote computing system 254 to a network interface 220 of the ambulatory medicament device 250 over an end-to-end wireless data connection. The end-to-end wireless data connection may be any type of wide area network connection. For example, the wide area network may be a cellular network and the end-to-end wireless data connection may be a cellular network connection (e.g., a 4G LTE or 5G network connection). The ambulatory medicament device 250 may receive the dose control signals via the end-to-end wireless data connection and may provide the dose control signals to the medicament pump 212 to administer medicament to a subject.

The electronic processor 204A, electronic processor 204B, and electronic processor 204C may collectively or individually be referred to as an electronic processor 204. It should be understood that with both a local device, such as the electronic device 252, or a remote device, such as the remote computing system 254, the electronic processor 204 may determine dosing information, which may be provided over a communication connection to the ambulatory medicament device 250. The ambulatory medicament device 250 may generate the dose control signals to control the medicament pump 212 based on the received dosing information. The dose control signals may be generated by a controller or processor included in the ambulatory medicament device 250 as illustrated with respect to the FIG. 2D.

FIG. 2D illustrates a block diagram of a fourth example of a glucose level control system 200D. The glucose level control system 200D may include a plurality of controllers distributed among different elements of the glucose level control system 200D that cooperate to generate a dose control signal to control the medicament pump 212 to administer medicament to a subject. For example, the glucose level control system 200D includes a controller 202A of the ambulatory medicament device 250, a controller 202B of the electronic device 252, and a controller 202C of the remote computing system 254, each of which may function independently or in conjunction with each other to generate dose control signals to control the medicament pump 212.

In the example glucose level control system 200D of FIG. 2D, the remote computing system 254 can transmit or receive data or instructions via the network interface 222, which may establish a network connection with a network interface 224 of the electronic device 252. The network connection may be any through any type of wide area network. For example, the network connection may be a cellular network or any other type of computing network that relies on shared protocols to establish communication between computing systems, such as the remote computing system 254 and the electronic device 252. Further, the electronic device 252 can transmit or receive data or instructions via the transceiver 216, which may establish a communication connection with a transceiver 214 of the ambulatory medicament device 250. The communication connection between the transceivers 214 and 216 may differ from that of the network connection between the network interfaces 222 and 224 in that the communication connection between the transceivers 214 and 216 may be implemented using one or more short-range or near-field communication protocols (e.g., Bluetooth®, Zigbee®, LoRa®, NFC, etc.) while the network connection between the network interfaces 222 and 224 may be implemented using one or more wide-area network protocols (e.g., Fiber-optic communication, cable modem, ISDN, etc.), which may be wired or wireless. In some cases, the wide-area network may include the Internet and/or one or more cellular networks. In certain embodiments, the transceivers 214 and 216 and the network interfaces 222 and 224 may implement the same protocols. In some such cases, the communication connection that may be established between the transceivers 214 and 216 may be of the same type as the network connection between the network interfaces 222 and 224.

In some embodiments, the electronic device 252 can be omitted. In some such cases, the controller 202A and the controller 202C may function independently or in conjunction with each other to generate dose control signals to control the medicament pump 212. In some such embodiments, the ambulatory medicament device 250 may include a network interface (not shown) to support a direct end-to-end wireless data connection, or other network connection, with the remote computing system 254.

The glucose level control system 200A, glucose level control system 200B, glucose level control system 200C, and glucose level control system 200D may collectively or individually be referred to as a glucose level control system 200. Further, in some embodiments, the glucose level control system 200 may be the ambulatory medicament device 250 or may be included as part of the ambulatory medicament device 250. Although illustrated as part of the ambulatory medicament device 250, the medicament pump 212 may be a separate device that is in communication with or capable of being controlled by the ambulatory medicament device 250 or the glucose level control system 200.

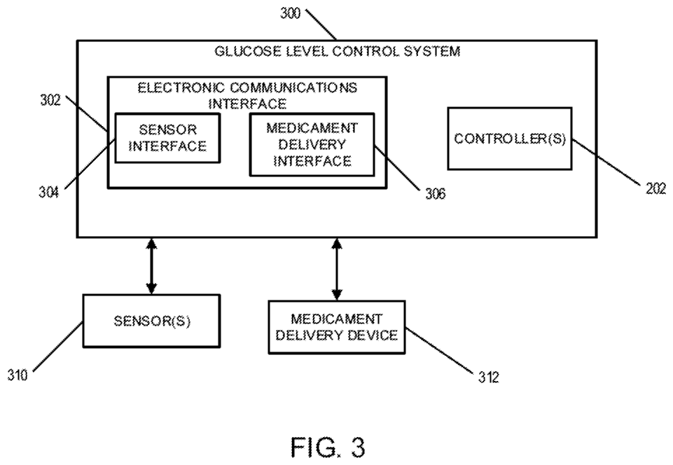

FIG. 3 illustrates a block diagram of an example glucose level control system 300 that includes an electronic communications interface 302. The glucose level control system 300 may include one or more of the embodiments described with respect to any of the glucose level control systems 200.

The electronic communications interface 302 may include any type of circuitry configured to send and receive electronic data from one or more electronic devices. The electronic communications interface 302 may include input/output (I/O) circuitry configured to interface with one or more electronic devices configured to facilitate glucose level control of a subject. For example, the electronic communications interface 302 may include a sensor interface 304 configured to receive a glucose level signal or glucose level data from a glucose level sensor 310, such as a continuous glucose monitor (CGM) sensor. Some CGM sensors may generate glucose level data or a corresponding glucose level signal at fixed measurement intervals, such as at one-minute, five-minute, or ten-minute intervals. The glucose level sensor 310 can be operatively connected to a subject in order to generate a glucose level signal that corresponds to a glucose estimate or measurement of a glucose level of the subject. It should be understood that the sensor interface 304 may interface or communicate with other types of sensors. For example, the glucose level sensor 310 may be replaced with or supplemented by a heart rate monitor.

As another example, the electronic communications interface 302 may include a medicament delivery interface 306. The medicament delivery interface 306 may be configured to provide a dose control signal to a medicament delivery device 312. The dose control signal may be generated by a controller 202 (which may represent one or more controllers) based at least in part on a glucose level signal. Alternatively, or in addition, the medicament delivery device 312 may provide an indication of a recommended dose of medicament to the medicament delivery device 312. The medicament delivery device 312 may include any type of device configured to administer (automatically or in response to user-interaction) medicament to a subject. For example, the medicament delivery device 312 may be or may include a medicament pump 212 or an insulin pen.

In some embodiments, the sensor interface 304 may communicate with the glucose level sensor 310, or other sensors, via a short-range wireless connection (e.g., using the transceiver 214). In some embodiments, the sensor can be an insulin level sensor that can detect insulin levels or any other type of analyte sensor. The sensor interface 304 can be configured to receive an insulin level signal from the sensor, which can correspond to an insulin level estimate or measurement of the subject (e.g., a concentration of insulin in the subject's blood). The insulin level signal can be used by the one or more controllers discussed herein to generate a dose control signal, which can be provided to the medicament pump 212 via the medicament delivery interface 306. In some embodiments, the sensor can include a glucose level sensor 310 and an insulin sensor.

In some embodiments, the medicament delivery interface 306 may communicate with the medicament delivery device 312 via a short-range wireless connection (e.g., using the transceiver 214). Alternatively, or in addition, the medicament delivery interface 306 may communicate with the medicament delivery device 312 via a local data bus, such as in implementations where the controller 202, the medicament delivery interface 306, and the medicament delivery device 312 are integrated into a single ambulatory medicament device 250. In some cases, the medicament delivery interface 306 may communicate with the medicament delivery device 312 via a wide area network using, for example, the network interface 220.

The controller 202 can be configured to generate a dose control signal using a control algorithm that generates at least one of a basal dose, a correction dose, and/or a food-intake or meal dose. Examples of control algorithms that can be used to generate these doses are disclosed in U.S. Pat. Nos. 7,806,854; 9,833,570; 10,543,313; 10,842,934; 10,940,267; U.S. Patent Publication No. 2021/0213200; and International or PCT Publication No. WO 2012/058694 (referenced herein as the “Controller Disclosures”), the entire contents of which are hereby incorporated by reference herein in their entirety and made a part of this specification. The correction dose can include regulatory (e.g., insulin or an insulin analog) or counter-regulatory agent (e.g., Glucagon) and can be generated using a model-predictive control (MPC) algorithm and/or a biexponential pharmacokinetic (PK) model. Examples of different algorithms and/or models that may be used to generate the correction doses are disclosed in the Controller Disclosures. The basal dose can include regulatory agent and can be generated using a basal control algorithm, such as those disclosed in the Controller Disclosures. The food-intake dose can include regulatory agent and can be generated using a meal control algorithm such as those disclosed in the Controller Disclosures. In some cases, the food-intake dose may be administered in response to any food-intake, food-intake of a particular size, or food-intake associated with particular defined meals. As indicated above, in some cases, the correction dose may include a counter-regulatory agent. However, in other cases, the counter-regulatory agent may be controlled or dosed separately from the correction dose. Additional aspects and improvements for at least some of these controllers are disclosed herein. The dose control signal can be transmitted to a medicament delivery device 312 via the electronic communications interface 302 (e.g., using the medicament delivery interface 306). Alternatively, or in addition, the dose control signal may be transmitted to a medicament pump 212 (or other medicament delivery device 312) via an electrical conductor in implementations where the controller 202 is integrated into the same housing as the medicament pump 212, which may include an infusion motor or other delivery components.

As indicated above, in some cases a glucose level sensor 310 can measure a glucose level of a subject. The control algorithm executed by the controller 202 can determine one or more doses of medicament based at least in part on the glucose level of the subject measured by the glucose level sensor 310 or by an isolated glucose level measurement provided to the controller 202. In some embodiments, the controller 202 may determine a medicament on board value. The medicament on board value may be used by the controller 202 instead of or in addition to the glucose level of the subject to generate a dose control signal using a control algorithm. In other words, in some embodiments, the controller 202 may generate a dose control signal based at least in part on the medicament on board value or the total medicament (e.g., insulin) that is within the subject or that is still active to a minimum degree.

The medicament on board value may correspond to a measurement or an accounting of the total medicament in the subject, in the subject's blood plasma, and/or that is active in the subject's body. The medicament on board value may be obtained by tracking medicament therapy provided to the subject. As different subjects may process medicament (e.g., insulin) differently, the determination of the amount of medicament on board may be a subject-specific determination. Accordingly, in some cases, the medicament on board value may be adjusted based on subject-specific parameters (e.g., weight or sex) or using a learning algorithm that may determine an average time of activity of the medicament for the specific subject using one or more measurements corresponding to the activity of the measurement (e.g., a measure of glucose level at one or more times subsequent to a dose of insulin). Further, as each subject may process medicament differently, in some cases, the medicament on board value may be an estimate of the total medicament within the subject and/or that remains active at a particular point in time. Further, the estimate of medicament on board may be determined based at least in part on total administered medicament, glucose level of the subject, food-intake announcements, activity level, and/or other subject-specific data that may affect the determination of medicament on board.

The medicament on board data value may be based at least in part on one or more basal doses and/or one or more non-basal dose (e.g., a food-intake dose, correction bolus, etc.). In some embodiments, the medicament on board data may correspond with the insulin on board of the subject over a therapy period.

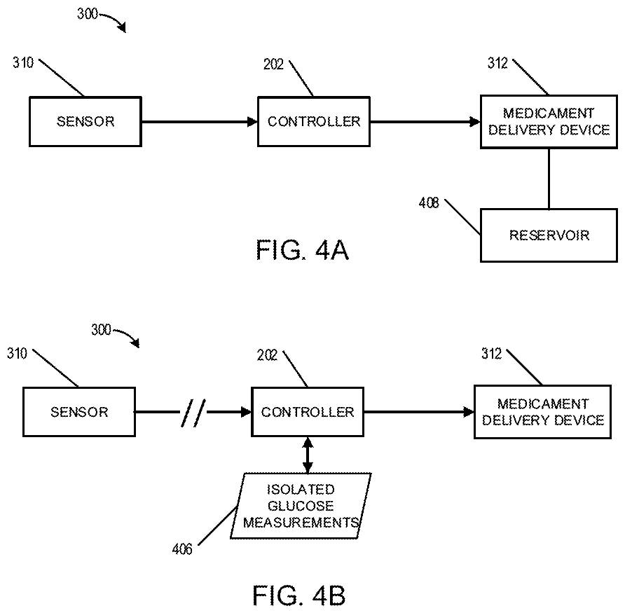

FIG. 4A illustrates a block diagram of an example of elements of a glucose level control system 300 operating in an “online mode.” In the online mode, the controller 202 receives a glucose level signal corresponding to a glucose level of a subject from the glucose level sensor 310. Further, during online mode, a control algorithm implemented by the controller 202 may generate a dose control signal that causes the medicament delivery device 312 to administer a medicament dose. The control algorithm may generate the dose control signal based at least in part on the glucose level signal received from the glucose level sensor 310. Further, the control algorithm may generate the dose control signal based at least in part on control parameters, or values of the control parameters, of the control algorithm. The medicament dose may be a correction dose, a basal dose, or a food-intake dose of medicament. The medicament delivery device 312 may be configured to deliver at least correction doses and basal doses to the subject without substantial or any user intervention when the controller 202 is operating in the online mode. In some examples, the medicament delivery device 312 can include one or more medicament cartridges or can have an integrated reservoir 408 of medicament. The reservoir 408 may be integrated with the medicament delivery device 312. A medicament stored in the reservoir 408 can be delivered to the subject by operation of the medicament delivery device 312. A pump motor of the medicament delivery device 312 can direct medicament for infusion in response to one or more dose control signals as discussed herein. In various embodiments, the operation of the medicament delivery device 312 can be controlled by the controller 202. In some cases, during the online mode, the controller 202 may generate the dose control signal using one or more control schemes described in the Controller Disclosures.

FIG. 4B illustrates a block diagram of an example of elements of a glucose level control system 300 operating in an “offline mode.” In the offline mode, the controller 202 do not receive a glucose level signal from the glucose level sensor 310. The glucose level signal may not be received from the glucose level sensor 310 for a number of reasons (as indicated by the break in the arrow connecting the glucose level sensor 310 block and the controller 202 block in FIG. 4B). For example, the glucose level sensor 310 may not be present or operatively connected to the subject. As another example, the glucose level sensor 310 may be damaged or a communication connection between the glucose level sensor 310 and the controller 202 may have failed or been interrupted. In yet another example, the glucose level sensor 310 may have at least partially become disconnected from the subject. Accordingly, in some cases, the omission of the glucose level signal may be intended and in other cases, the omission of the glucose level signal may be unintended. In other words, in some cases, the glucose level signal may be expected, but not received.

In the offline mode, a control algorithm implemented by the controller 202 may generate a dose control signal that causes the medicament delivery device 312 to administer a medicament dose. However, while the controller 202 may use a glucose level signal received from the glucose level sensor 310 to generate the dose control signal in online mode, in offline mode, the controller 202 may base the dose control signal on an isolated glucose measurement 406 (or a plurality of isolated glucose measurements). The isolated glucose measurements 406 may be based on glucose test strips or other manual measurement technology for obtaining glucose level data corresponding to a glucose level of the subject. In the offline mode, the controller may generate dose control signals using one or more algorithms described in the Controller Disclosures. In the offline mode, the control algorithm may generate a dose control signal that implements correction doses in response to isolated glucose measurements 406 (such as, for example, measurements obtained from the subject using glucose test strips). Alternatively, or in addition, the dose control signals may be generated based at least in part on insulin measurements. Further, the dose control signals may be based on control parameters of the control algorithm. The medicament delivery device 312 may be configured to deliver basal doses to the subject without substantial user intervention and can deliver correction doses to the subject in response to isolated glucose measurements 406 and/or isolated insulin measurements while the controller 202 remains in offline mode. In some embodiments, the controller 202 may operate in “offline mode” during time periods when one or more glucose level signals received from the glucose level sensor 310 are identified as artifacts (e.g., suspected artifacts) as described in PCT Application No. PCT/US2022/027531, the entire contents of which are hereby incorporated by reference in its entirety herein.

Further, as in online mode, during offline mode the control algorithm may generate the dose control signal based at least in part on control parameters, or values of the control parameters, of the control algorithm. Further, as with the online mode, the medicament dose may be a correction dose, a basal dose, or a food-intake dose of medicament. And the medicament delivery device 312 may be configured to deliver at least correction doses and basal doses to the subject without substantial or any user intervention when the controller 202 is operating in the offline mode. However, in contrast to the online mode, additional user intervention may be required to provide the isolated glucose measurements 406 to the controller 202 when operating in the offline mode. In some cases, additional user intervention may not be required during offline mode because, for example, the controller 202 may generate a dose control signal based on glucose level data obtained when the glucose level control system 300 was operating in the online mode or from previously obtained isolated glucose measurements 406. In some such cases, additional user intervention may not be required for a period of time when operating in offline mode. However, after the period of time has elapsed, additional user intervention, such as providing isolated glucose measurements 406 to the controller 202, may be required.

Regardless of whether the glucose level control system 300 is operating in online mode or offline mode, the glucose level control system 300 can operate in an open-loop mode or a closed-loop mode. When operating in the open-loop mode, the glucose level control system 300, using a control algorithm, determines the dose control signal based on one or more inputs to the glucose level control system 300. The one or more inputs may include a glucose level signal and/or one or more isolated glucose measurements 406. Further, the one or more inputs may include an indication of food-intake or characteristics of the food consumed during a food-intake event. In some cases, the one or more inputs may include insulin sensitivity of the subject, characteristics of the insulin absorption of the subject, characteristics of the medicament being administered, or any other characteristics of the subject or medicament that may affect an output of the control algorithm.

When operating in closed-loop mode, the glucose level control system 300, using a control algorithm, determines the dose control signal based on one or more prior outputs of the control algorithm. For example, the dose control signal may be based on prior determined dose control signals or corresponding medicament doses. In some cases, the glucose level control system 300 may determine the dose control signal based on a combination of the prior dose control signals and one or more inputs to the glucose level control system 300, which may include one or more of the inputs described with respect to open-loop mode.

### Example Glucose Level Control System Implementation

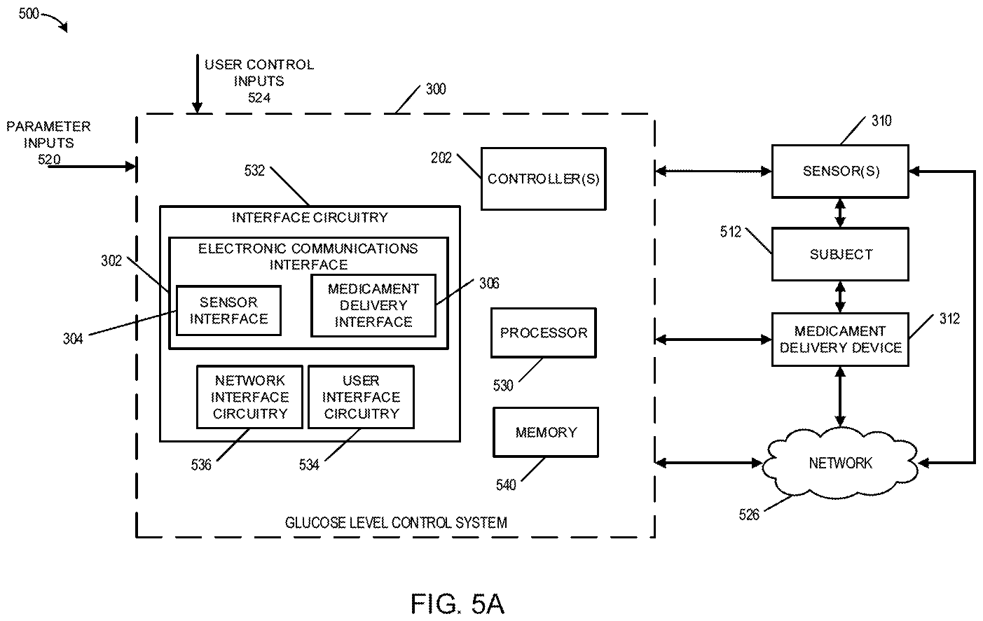

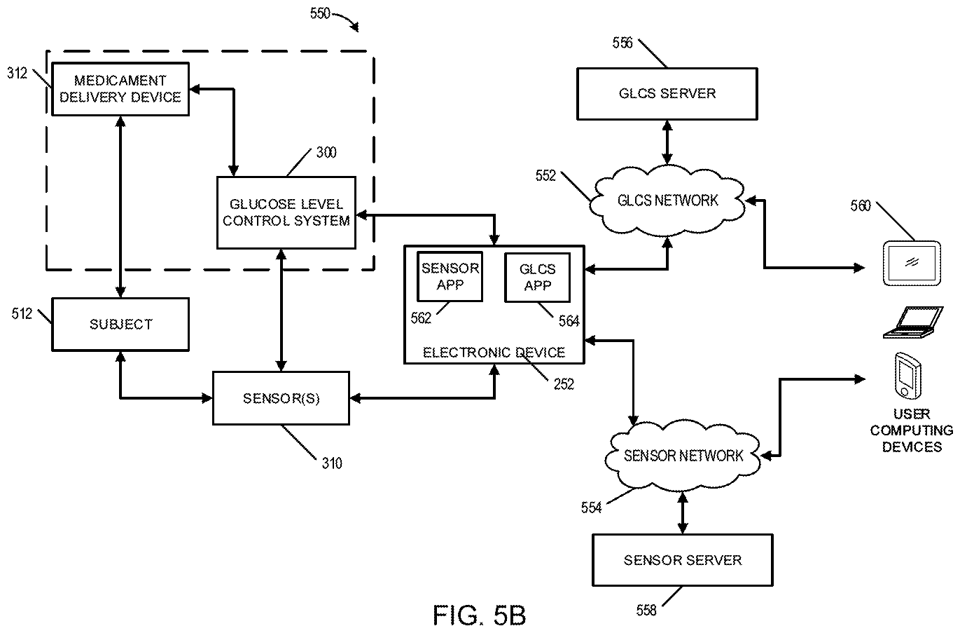

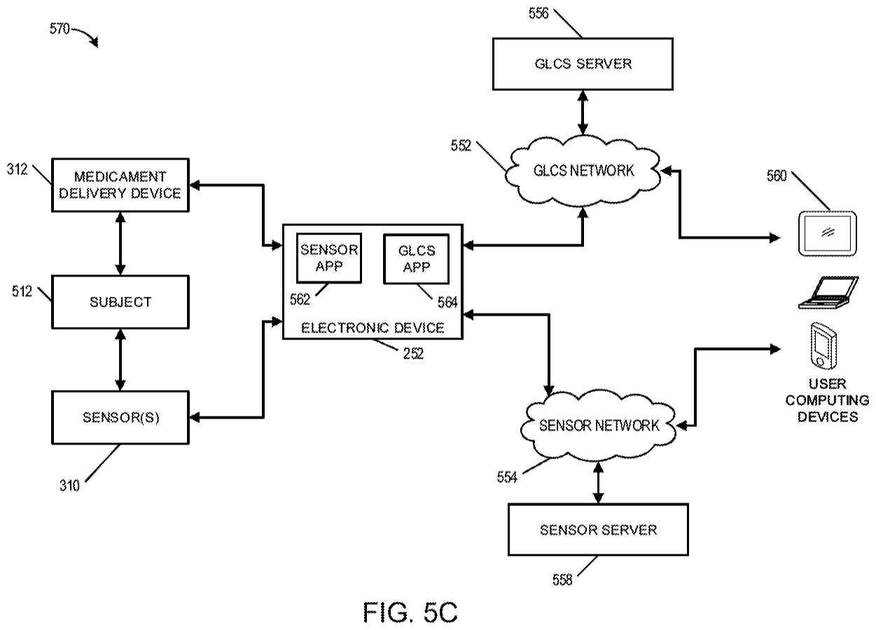

FIG. 5A illustrates a block diagram of an example glucose level control system environment 500 that includes a glucose level control system 300 in accordance with certain embodiments. The glucose level control system 300 can regulate a glucose level (e.g., blood glucose level) of a subject 512. Typically, the subject 512 is a human subject. However, it is possible to adapt the glucose level control system 300 to regulate the glucose level of non-human animals. The glucose level control system 300 may be an example of an automated glucose level regulation system that can regulate the glucose level of the subject 512 with little or no user interaction with the glucose level control system 300. In some cases, a user may interact with the glucose level control system 300 to initialize, setup, or reconfigure the glucose level control system 300, but little to no further user interaction may occur during operation. Alternatively, or in addition, the glucose level control system 300 may be configured to provide manual regulation of the glucose level of the subject. In some cases, the glucose level control system 300 may perform both automatic and manual regulation of the glucose level of the subject. For example, basal and correction doses of medicament may be controlled automatically by the glucose level control system 300 while food-intake doses may be at least partially controlled manually or in response to user interaction with the glucose level control system 300.

The glucose level control system 300 may cause one or more medicament delivery devices 312 to administer one or more doses of insulin to the subject 512. The medicament delivery device 312 may include a medicament pump 212 that can be coupled by a catheter to a subcutaneous space of the subject 512. In some cases, the medicament delivery device 312 may include an insulin pen or medicament patch (e.g., a patch pump), or any other type of device that may administer medicament to the subject 512. As illustrated, the medicament delivery device 312 may be separate, but in communication with the glucose level control system 300. However, in some cases, the medicament delivery device 312 and the glucose level control system 300 may be integrated.

As indicated above, the medicament delivered by the medicament delivery device 312 may be insulin. Alternatively, or in addition, the medicament delivery device 312 may deliver a counter-regulatory agent or hyperglycemic agent, such as glucagon or dextrose, for control of the glucose level under certain circumstances. For the delivery of both insulin and a counter-regulatory agent (e.g., glucagon), the medicament delivery device 312 may be a mechanically driven infusion mechanism having dual cartridges for insulin and the counter-regulatory agent, respectively. In the present description, reference is made to glucagon specifically, but it is to be understood that this is for convenience only and that other counter-regulatory agents (e.g., dextrose) may be used. Similarly, the term “insulin” herein is to be understood as encompassing all forms of insulin-like substances including natural human or animal insulin as well as synthetic insulin in any of a variety of forms (sometimes referred to as “insulin analogs”).

During online and/or autonomous operation, a glucose level sensor 310 may be operatively coupled to the subject 512. The glucose level sensor 310 may continually sample a glucose level of the subject 512 and the glucose level sensor 310 may be referred to as a continuous glucose monitoring (CGM) sensor. The glucose level sensor 310 may continuously or periodically measure or sense glucose levels of the subject 512 for at least a period of time. The measured or sensed glucose levels of the subject 512 may be blood glucose levels and/or glucose levels of other parts of the subject 512, such as interstitial fluid. Sensing may be accomplished in a variety of ways, generally involving some form of physical coupling between the subject 512 and the glucose level sensor 310.

A controller 202 may control operation of the medicament delivery device 312 using a control algorithm. The control algorithm may be implemented or executed as a function of a glucose level signal received from the glucose level sensor 310. Further, the control algorithm may be subject to one or more control parameters that affect the execution and/or output of the control algorithm. In some cases, one or more control parameters may include or may be subject to one or more parameter inputs 520, which may be programmed and/or provided by a user, such as the subject 512, a parent or guardian of the subject 512, or a healthcare provider (e.g., a clinician or doctor). The parameter inputs 520 may include any type of control parameter that can affect implementation or execution of the control algorithm by the controller 202. One example parameter input 520 may include the weight or mass of the subject 512.

In some cases, the glucose level control system 300 can provide effective automated glucose level control without receiving explicit information regarding either meals (or other food-intake) that the subject 512 has ingested or any other “feedforward” information, which may be achieved in part by an adaptive aspect to operation of the controller 202. In other cases, the glucose level control system 300 can use received information regarding either meals (or other food-intake) that the subject 512 ingested, or plans to ingest, or other “feedforward” information to modify control of the glucose level of the subject 512 and/or the delivery of medicament including insulin or counter-regulatory agent.

The controller 202 may be electronic device with control circuitry that provides operating functionality as described herein. In some embodiments, the controller 202 may be implemented using application specific hardware that can execute one or more computer programs including a set of computer instructions. Alternatively, the controller 202 may be implemented using a general-purpose hardware processor configured to execute computer-executable instructions corresponding to one or more computer programs each including respective sets of computer instructions.

In some cases, the glucose level control system 300 may include one or more processors 530, memory 540, and interface circuitry 532. The one or more processors 530, memory 540, and interface circuitry 532 may communicate through a bus (e.g., a set of wires or conductive traces) or other internal communications system that can transfer data or instructions between components of a computer system. The one or more processors 530 may execute one or more sets of computer-executable instructions stored in the memory 540 and configured to control execution of the glucose level control system 300. The one or more processors 530 may control operations and features of the glucose level control system 300 that are separate from the control algorithm configured to maintain or control the glucose level of the subject 512. Alternatively, or in addition, the one or more processors 530 may execute the control algorithm. In such cases, the controller 202 may be integrated with the one or more processors 530 and/or the one or more processors 530 may perform the functionality of the controller 202 and a separate controller 202 may be omitted. The one or more processors 530 may be or may include any type of general purpose processor including, but not limited to a central processing unit (“CPU”), a graphics processing unit (“GPU”), a complex programmable logic device (“CPLD”), a field programmable gate array (“FPGA”), or an application-specific integrated circuit (“ASIC”).

The memory 540 may communicate computer-executable instructions to the one or more processors 530 for execution. The computer-executable instructions may be communicated to the one or more processors 530 via a bus, a cache memory scheme, a direct connection or any other internal communications system. Further, the memory 540 may be further configured to store data associated with operation of the glucose level control system 300 and/or therapy delivered to the subject 512. For example, the memory 540 may store the timing and amount of medicament (e.g., insulin) delivery. The memory 540 can include any type of non-volatile memory and/or volatile memory, such as RAM. The non-volatile memory may include flash memory or solid-state memory. In some cases, the memory 540 may include both main memory (e.g., RAM, ROM, cache, etc.) or primary memory that can store instructions and data actively in use by the one or more processors 530 and long term storage or secondary memory (e.g., magnetic disk memory, optical disk memory, flash, or solid-state memory) that can store applications or data permanently or over a longer term time period than the main memory. While the storage may maintain instructions and data when the glucose level control system 300 is unpowered, the main memory may maintain instructions and data only so long as the glucose level control system 300 is powered.

The interface circuitry 532 can include any type of input/output circuitry that can provide one or more interfaces for interacting with and/or communicating with the glucose level control system 300. For example, the interface circuitry 532 may include the previously described electronic communications interface 302. As previously described, the electronic communications interface 302 may include a sensor interface 304 that can communicate with or receive input from the glucose level sensor 310 and a medicament delivery interface 306 that can communicate with or output commands to the medicament delivery device 312.

The interface circuitry 532 can further include network interface circuitry 536 that enables communication with one or more electronic devices using one or more communication protocols. The interface circuitry 532 can include the transceiver 214, the network interface 220, or any other communication circuitry that enables the glucose level control system 300 to communicate via a wired or wireless connection with an electronic device 252, a remote computing system 254, or any other device. For example, the interface circuitry 532 may include one or more network interface cards and/or wireless radios including, but not limited to, a Bluetooth radio, a Bluetooth Low Energy (BLE) radio, a cellular radio (e.g., a 4G LTE transceiver, a 5G transceiver, or a ND-LTE radio, and the like), an RFID reader, or any other near field, short-range, or wide-area communications circuitry.

As described, the glucose level control system 300 may communicate with one or more of a glucose level sensor 310, a medicament delivery device 312, an electronic device 252, and/or a remote computing system 254, using wired or wireless communication. In some cases, the glucose level control system 300 may, using network interface circuitry 536 and/or the electronic communications interface 302, communicate directly with one or more devices including, but not limited to, the aforementioned devices (e.g., the electronic device 252 or the remote computing system 254). Alternatively, or in addition, the glucose level control system 300 may communicate with the one or more devices via a network 526.

The network 526 may include any type of wired or wireless communication network, including a combination of wired and wireless communication networks. For example, the network 526 may include a local area network (LAN), a wide area network (WAN), a cellular network (e.g., a 4G LTE network, a 5G network, etc.), a wireless LAN (WLAN), an Internet-of-Things (IOT) network, or a combination of networks. Further, in some cases, the network 526 may include the Internet.

In some cases, the interface circuitry 532 may include user interface circuitry 534, which may include any circuitry or processors that may output a user interface to a user and/or receive user input from the user via the user interface. The user interface circuitry 534 may receive one or more signals from a processor 530 corresponding to a user interface. The user interface circuitry 534 may control a display to present the user interface to a user based on the one or more signals received from the processor 530. Further, the user interface circuitry 534 may include any circuitry that can receive a signal corresponding to an interaction by a user with a user interface and can provide the signal to the processor 530 and/or controller 202 for further processing.

The user interface circuitry 534 may include any type of output device (e.g., a display screen, audio system, lights, haptic circuitry, etc.) for providing output. Further, the user interface circuitry 534 may include any type of input device (e.g., buttons, dials, keypads, switches, etc.) for receiving input. In some cases, the user interface circuitry 534 may include combined input and output devices, such as a touchscreen controller for controlling a touchscreen or other touch-sensitive device configured to output data and receive input. The touchscreen controller may generate user input signals corresponding to user inputs, such as user control inputs 524. Further, the user interface circuitry 534 can cause user interface screens to be displayed on a display (e.g., a touchscreen display or non-touchscreen display) of the glucose level control system 300. The touchscreen display may accept input via capacitive touch, resistive touch, or other touch-sensitive technology. The user interface circuitry 534 can register the position of one or more touches on a surface of a touch-sensitive display. Further, the user interface circuitry 534 may determine different types of touches or gestures on the touch-sensitive display including, but not limited to, multitouch gestures, taps, multitap inputs (e.g., double or triple taps, etc.), swipes, press and hold interactions, etc.

As described, the user interface circuitry 534 may receive user input in response to interaction with a touchscreen or other touch-sensitive device. Alternatively, or in addition, the user interface circuitry 534 may receive user input via non-touchscreen devices. For example, the glucose level control system 300 may include an alphanumeric keypad (which may include letters, numbers, symbols, or other keys), one or more physical buttons, one or more switches, one or more knobs, or any other type of user interface element that enables a user to interact with the glucose level control system 300. The user interface circuitry 534 may process one or more signals or inputs received via the one or more user interface elements of the glucose level control system 300, or user interface elements of another device (e.g., an electronic device 252) in communication with the glucose level control system 300. In some embodiments, the user interface circuitry 534 may include a sleep and/or wake feature that enables a user to place the glucose level control system 300 into a sleep mode or to wake from a sleep mode. The sleep mode may deactivate a user interface and/or one or more additional features of the glucose level control system 300 enabling a reduction in power consumption and/or preventing inadvertent changes to a configuration of the glucose level control system 300.

In some embodiments, the interface circuitry 532 may include an audio or voice recognition system (e.g., a speaker, a microphone, an interactive voice response (IVR) system, or the like) for receiving an audio or voice command, or other utterance from a user. In some cases, the user interface circuitry 534 may be configured to output via a speaker a sound or voice that may be prestored or generated in real time.

In some implementations, one or more of the interfaces may be provided by the one or more processors 530 or may be provided by the interface circuitry 532 in conjunction with the one or more processors 530. Further, in some cases, one or more of the interfaces (e.g., a user interface) may be provided by or in conjunction with an electronic device 252 or a remote computing system 254. For example, a user (e.g., the subject 512, a parent or guardian, or a clinician or other healthcare provider) may interact with a smartphone, a smartwatch, a tablet, a desktop, or a laptop, and the like, to provide input to the glucose level control system 300 or to access output generated by the glucose level control system 300.

User interaction with the glucose level control system 300 may be performed by the subject 512. Alternatively, or in addition, user interaction may be performed by a user that is separate from the subject 512. For example, a parent or guardian may interact with the glucose level control system 300 on behalf of a child or other subject that may not be capable of configuring the glucose level control system 300 without assistance. In another example, a healthcare provider may assist the subject 512 in configuring the glucose level control system 300 based on subject-specific parameters and/or to help train the subject 512 in maintaining his or her disease.

In some cases, the controller 202 may perform some or all of the functionality of the glucose level control system 300. In some such cases, the processor 530 may be optional or omitted. In other cases, the controller 202 may at least perform automated glucose level control of the subject 512 and/or may generate recommended medicament doses to facilitate manual glucose level control or hybrid glucose level control that includes both automated and manual glucose level control of the subject 512, while one or more separate processors 530 may perform one or more additional operations of the glucose level control system 300. These additional operations may include any features the glucose level control system 300 can provide in addition to the glucose level control provided by the controller 202. For example, the additional operations may include tracking or logging of occurrences of hyperglycemic or hypoglycemic events or risk events, outputting data to a user (e.g., the subject 512, a parent or guardian, or a clinician or other healthcare provider), controlling or initiating communication with another computing system (e.g., the electronic device 252 or the remote computing system 254), regulating access to the glucose level control system 300, or other operations unrelated to operation of a medicament pump 212 or the medicament delivery device 312.

As described above, the controller 202 is capable of operating in an online mode in which the controller 202 receives a glucose level signal corresponding to a glucose level of a subject 512 from the glucose level sensor 310. Alternatively, or in addition, the controller 202 can operate in an offline manner in which the controller 202 does not receive the glucose level of the subject 512 from the glucose level sensor 310. In some such cases, the controller 202 may cause the medicament delivery device 312 to deliver insulin (and potentially glucagon as well) independent of or without receipt of glucose levels from the glucose level sensor 310. In offline mode, the controller 202 may determine medicament doses based at least in part on isolated glucose measurements 406, past glucose level measurements from the glucose level sensor 310, historical therapy data (e.g., past medicament delivery), user input (e.g., food-intake announcements or manual medicament requests), or other control information that may be used to facilitate automatic operation of the glucose level control system 300 for at least a period of time. In some cases, the controller 202 may cause the medicament delivery device 312 to automatically deliver medicament for at least a period of time when operating in the offline mode. Alternatively, or in addition, the controller 202 may output a recommendation of medicament delivery when operating in the offline mode enabling a user to determine whether to deliver the recommended medicament (e.g., insulin, insulin analog, or glucagon) or to confirm whether delivery by the medicament delivery device 312 is to proceed. In some cases, the controller 202 may cause automatic medicament delivery for a period of time and subsequent to the period of time may cease providing automatic delivery of medicament. Cessation of automatic delivery of medicament may occur because, for example, data available for determining medicament delivery may become stale or too old to safely determine medicament dosing.

The offline operating mode of the glucose level control system 300 may be triggered in response to determining that the glucose level sensor 310 needs replacing, is not properly connected to the subject 512, is defective, that a signal is not being received from the glucose level sensor 310, or that the glucose level sensor 310 is absent. Further, in some cases, offline mode may occur in response to a user command, a determination of a lack of available medicament, or a malfunction of the glucose level control system 300. In some embodiments, operation of the glucose level control system 300 may be divided between online periods each including a succession of sampling intervals when a glucose level signal is available, and offline periods each including a succession of sampling intervals when the glucose level signal is either completely or intermittently unavailable. Although “online” and “offline” modes are generally defined by the availability of the glucose level signal, in some cases, offline operation may be user-selected even when a glucose level signal is available to the glucose level control system 300.

In some embodiments, a user (e.g., a subject 512, parent or guardian, healthcare provider, or any other user who may be authorized to help manage therapy of the subject 512, etc.) may provide one or more user control inputs 524 to the glucose level control system 300. The user control inputs 524 may be provided via a user interface generated by the user interface circuitry 534 or a user interface of another device (e.g., an electronic device 252 or remote computing system 254) in wired or wireless communication with the glucose level control system 300. The user control inputs 524 may be provided via a local or remote user interface. In some embodiments, the user interface may resemble that of an insulin pump or similar devices, e.g., by including control buttons for commanding the delivery of a medicament bolus and/or a display. In some embodiments, the glucose level control system 300 may have a wired or wireless interface to a remote device (e.g., an electronic device 252 or a remote computing system 254) that may incorporate a full-function user interface, such as a smartphone, smartwatch, laptop computer, desktop computer, cloud computing service, or other wearable device or computing device. In some embodiments, the wireless interface may provide access to a local area network, such as a personal home network, a company network, or otherwise. Alternatively, or in addition, the wireless interface may provide a direct connection between local devices available to a user (e.g., via Bluetooth or other near field communication technologies). In some cases, the wireless interface may provide access to a wide area network, such as, but not limited to, the Internet. For example, the wireless interface may include a cellular interface that permits access to a network via a 4G or 5G cellular connection. In some cases, the cellular interface may be a low power interface, such as narrowband LTE or other Internet of Things (IoT) interfaces.

As previously described, the one or more parameter inputs 520 may include any input that modifies or corresponds to a control parameter used by the controller 202 to execute a control algorithm for determining whether to administer medicament, the quantity of medicament to administer to the subject 512, and/or the timing of medicament delivery. The user control inputs 524 may include any type of input relating to additional features or control of the glucose level control system 300. In some cases, the user control inputs 524 may include inputs that cause or relate to operation of the controller 202. For example, the user control inputs 524 may include inputs relating to food-intake or a manual request for medicament delivery. Additional non-limiting examples of the user control inputs 524 may include inputs relating to alarm settings, security settings, authorization of one or more users to access data or features of the glucose level control system 300, data delivery configurations, medicament cartridge installation, site changes or setup for connecting the medicament delivery device 312 to the subject 512, user interface preferences, location settings, or any other configuration options of the glucose level control system 300.

In an offline mode discussed herein, the glucose level sensor 310 may be absent, non-functioning, or not operatively coupled to the subject 512. As such, in the offline mode, the glucose level signal may not be available to control automatic operation. In some cases, a user may provide one or more glucose level measurements to the glucose level control system 300 to facilitate automatic operation of the glucose level control system 300. These measurements may be provided over a particular time period. Alternatively, or in addition, the glucose level control system 300 may use a therapy history and/or a history of prior glucose level control measurements to facilitate automatic operation of the glucose level control system 300 for at least a particular time period.

FIG. 5B illustrates a block diagram of a second example glucose level control system environment 550 in accordance with certain embodiments. As with the glucose level control system environment 500, the glucose level control system environment 550 includes a glucose level control system 300. Further, the glucose level control system environment 550 includes a medicament delivery device 312. The glucose level control system 300 may communicate with the medicament delivery device 312 enabling the glucose level control system 300 to control medicament delivery by the medicament delivery device 312 to the subject 512. Further, the glucose level control system 300 may obtain therapy data (e.g., a size of medicament delivered, status of a medicament cartridge, etc.) from the medicament delivery device 312. As illustrated in glucose level control system environment 550, the glucose level control system 300 and the medicament delivery device 312 may be separate devices. Alternatively, the glucose level control system 300 may be incorporated as part of the medicament delivery device 312 or vice versa, the medicament delivery device 312 may be incorporated as part of the glucose level control system 300. Thus, optionally, the glucose level control system 300 and the medicament delivery device 312 may be replaced by a single system as illustrated by the dashed line box.

The glucose level sensor 310 may be operatively connected to the subject 512 and may measure a glucose level of fluid (e.g., blood or interstitial fluid) of the subject 512. The glucose level sensor 310 may generate a signal corresponding to the measured glucose level and may provide the signal to the glucose level control system 300, which may control the medicament delivery device 312 based at least in part on the sensor signal.

In some embodiments, one or both of the glucose level control system 300 and the glucose level sensor 310 may communicate with an electronic device 252. For example, the glucose level sensor 310 may provide sensor data (e.g., glucose level data) to the electronic device 252. As another example, the glucose level control system 300 may provide to the electronic device 252 historical therapy data corresponding to therapy provided to the subject 512 by the glucose level control system 300 and/or medicament delivery device 312. The electronic device 252 may include any type of computing device that can communicate directly or via a wired or wireless network with the glucose level control system 300. Further, in some cases, the electronic device 252 may include any type of computing device that can execute one or more computer-executable instructions or applications that can control, at least in part, the glucose level control system 300 and/or the medicament delivery device 312. For example, the electronic device 252 may include a smartphone, a smartwatch, a tablet, a laptop, a pair of smartglasses, an application specific controller, and the like.

In some embodiments, the electronic device 252 may host one or more applications that may facilitate accessing data of one or more of the glucose level sensor 310, the glucose level control system 300, and the medicament delivery device 312. Further, the one or more applications may enable the electronic device 252 to at least partially control one or more of the glucose level sensor 310, the glucose level control system 300, and/or the medicament delivery device 312. For example, the electronic device 252 may include a sensor application 562 and/or a glucose level control system (GLCS) application, which may be referred to as a GLCS application 564. In some cases, the sensor application 562 and the GLCS application 564 may be capable of communicating with each other. Alternatively, the sensor application 562 and the GLCS application 564 may be incorporated into a single application. In other words, one application may implement the features described herein of both the sensor application 562 and the GLCS application 564.

The sensor application 562 may include any application that can receive data from the glucose level sensor 310. For example, the sensor application 562 may receive a glucose level signal from the glucose level sensor 310 enabling the sensor application 562 to determine a corresponding glucose level of the subject 512 operatively connected to the glucose level sensor 310. The sensor application 562 may display, or cause to be displayed by the electronic device 252, the data received from the glucose level sensor 310 (e.g., a glucose level signal) and/or data corresponding to the received data (e.g., a glucose level). Further, the sensor application 562 may cause the electronic device 252 to transmit the raw data received from the glucose level sensor 310 and/or data determined from the raw data to a third-party system, such as a sensor server 558. The data may be transmitted to the sensor server 558 via a sensor network 554.

The sensor server 558 may include any system that can receive and/or store data obtained from the sensor application 562. The data may correspond to sensor readings of the glucose level sensor 310 and/or data generated by the sensor application 562 in response to sensor readings by the glucose level sensor 310. For example, the data may include glucose level signals, glucose levels, sensor status information (e.g., errors, length in use, remaining expected usage time, etc.) associated with the glucose level sensor 310, a type or model of the glucose level sensor 310, or any other data that may be obtained by the glucose level sensor 310 and/or associated with operation of the glucose level sensor 310.

The sensor network 554 can include any type of computing or communications network that enables communication between the electronic device 252 and the sensor server 558. In some cases, the sensor network 554 may be or may include a third-party network, such as a network that may be associated with a third-party that manages or maintains the sensor server 558. Alternatively, or in addition, the sensor network 554 may include the Internet and/or the network 526. In some cases, the electronic device 252 may access the sensor network 554 via the network 526 and/or the Internet.

The GLCS application 564 may include any application that can receive data from the glucose level control system 300. The data may include any type of data that may be determined by the glucose level control system 300 including, but not limited to, data associated with therapy provided by the medicament delivery device 312 and data associated with maintaining the disease of the subject 512. For example, the data may include therapy data associated with the timing and quantity of medicament administered to the subject 512. Further, the data may include data associated with the status of the subject 512 and/or the diseases of the subject 512, data associated with the status of the medicament delivery device 312 and/or the glucose level control system 300, and any other data that may be generated or maintained by the glucose level control system 300 and/or the medicament delivery device 312. In some cases, the data may include data obtained from the glucose level sensor 310. In other words, in some cases, the GLCS application 564 may obtain data from the glucose level sensor 310 via the sensor application 562, the glucose level sensor 310 directly, and/or via the glucose level control system 300.

In some cases, the GLCS application 564 may be further configured to control at least in part the glucose level control system 300, which may in turn control the medicament delivery device 312. Alternatively, or in addition, the GLCS application 564 may implement at least some of the features of the glucose level control system 300. In some such cases, the GLCS application 564 may function as the glucose level control system 300. In such cases, the GLCS application 564 may directly control the medicament delivery device 312. In other words, the GLCS application 564 may serve as the glucose level control system 300 and may communicate with the medicament delivery device 312 via the electronic device 252.

Further, the GLCS application 564 may display, or cause to be displayed by the electronic device 252, the data received from the glucose level control system 300 and/or data corresponding to the received data. Moreover, the GLCS application 564 may receive and/or display similar data as the sensor application 562 (e.g., sensor data received from the glucose level sensor 310). In addition, much like the sensor application 562, the GLCS application 564 may cause the electronic device 252 to transmit the raw data received from the glucose level control system 300 and/or data determined from the raw data to a third-party system, such as a GLCS server 556. The data may be transmitted to the GLCS server 556 via a GLCS network 552. In some cases, the sensor application 562 and the GLCS application 564 may be combined or part of the same application. In such cases, the combined application may be capable of communicating with one or both of the GLCS server 556 and the sensor server 558 via the sensor network 554, the GLCS network 552, and/or the network 526.

The GLCS server 556 may include any system that can receive and/or store data obtained from the GLCS application 564. The data may correspond to data generated by the medicament delivery device 312, the glucose level control system 300, the GLCS application 564, the glucose level sensor 310, the sensor application 562 and/or the GLCS application 564. For example, the data may include glucose level signals, glucose levels, sensor status information (e.g., errors, length in use, remaining expected usage time, etc.) associated with the glucose level sensor 310, a type or model of the glucose level sensor 310, therapy data (e.g., timing or quantity of medicament doses), status information associated with the medicament delivery device 312, status information associated with the glucose level control system 300, user interaction history with the glucose level control system 300, the medicament delivery device 312, the glucose level sensor 310, the sensor application 562, or the GLCS application 564, or any other data that may be obtained by the glucose level sensor 310, the medicament delivery device 312, the glucose level control system 300, the sensor application 562, and/or the GLCS application 564.

The sensor network GLCS network 552 can include any type of computing or communications network that enables communication between the electronic device 252 and the sensor application 562. In some cases, the GLCS network 552 may be or may include a third-party network, such as a network that may be associated with a third-party that manages or maintains the GLCS server 556. Alternatively, or in addition, the GLCS server 556 may include the Internet and/or the network 526. In some cases, the electronic device 252 may access the GLCS server 556 via the network 526 and/or the Internet.

In some embodiments, the sensor network 554 and the GLCS network 552 may be the same network. Further, in some cases, the sensor server 558 and the GLCS server 556 may be managed by the same party. Further, one or more of the sensor server 558, the GLCS server 556, the glucose level sensor 310, the medicament delivery device 312, the glucose level control system 300, the electronic device 252, the sensor application 562, and/or the GLCS application 564 may be managed, developed, and/or produced by the same party.

In some cases, the GLCS server 556 and/or the sensor server 558 may provide access to data provided by the electronic device 252 and/or data generated based at least in part on the data provided by the electronic device 252. The data access may be provided to the subject 512 and/or to other users associated with the subject 512 and/or authorized to access data associated with the subject 512. For example, parents, guardians, healthcare providers, or other authorized parties may access the data provided to or generated by the GLCS server 556 or the sensor server 558 using a user computing device 560. The user computing device 560 may include any type of computing device capable of accessing the GLCS server 556 and/or the sensor server 558 via a network, such as the sensor network 554, the GLCS server 556, the network 526, and/or the Internet.

Advantageously, the ability of authorized users (e.g., a parent, guardian, or healthcare provider) to access the GLCS server 556 and/or the sensor server 558 enables the authorized users to help monitor and maintain the disease and/or therapy of the subject 512. Moreover, the authorized users can determine whether the subject 512 requires assistance to help manage the disease of the subject 512.

FIG. 5C illustrates a block diagram of a third example glucose level control system environment 570 in accordance with certain embodiments. In contrast to the glucose level control system environment 550, the glucose level sensor 310 of the level control system environment 570 communicates with the electronic device 252 without communicating with the medicament delivery device 312. In some such cases, the electronic device 252 may communicate information or data (e.g., a glucose level signal or data derived from the glucose level signal) with the medicament delivery device 312 enabling the medicament delivery device 312 to provide therapy based at least in part on the measurements made by the glucose level sensor 310. Alternatively, or in addition, the electronic device 252 using, for example, the GLCS application 564 may generate control signals based at least in part on the glucose level sensor 310 measurements and may transmit the control signals to the medicament delivery device 312 thereby enabling the electronic device 252 to control the medicament delivery device 312 based at least in part on measurements of the glucose level sensor 310. In other words, in certain embodiments, the medicament delivery device 312 may be controlled based at least in part on sensor measurements of the subject 512 by the glucose level sensor 310 without the glucose level sensor 310 communicating directly with the medicament delivery device 312. Advantageously, communication or control via the electronic device 252 enables the use of complex control algorithms or processes to control a medicament delivery device 312 (e.g., a patch pump) that may have limited or no computer processing capabilities.

### Example Controller for a Glucose Level Control System

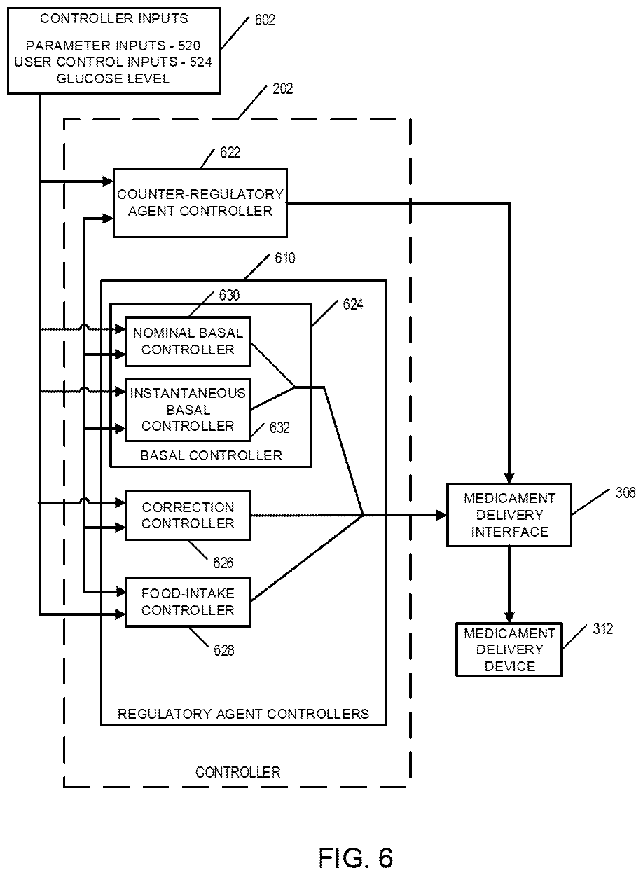

FIG. 6 illustrates a block diagram of an example controller 202 in accordance with certain embodiments. The controller 202 illustrated in FIG. 6 may represent a physical structure of different controllers or processors, or a logical structure that is implemented by one or more physical processors. In other words, a single processor may be used to implement each of the controllers illustrated in FIG. 6, each controller may be implemented by its own processor, or certain processors may implement multiple, but not necessarily all, of the controllers illustrated in FIG. 6 as part of the controller 202. Further, as illustrated in FIG. 6, each of the controllers may be implemented or part of the controller 202. Thus, to simplify discussion and not to limit the present disclosures, the counter-regulatory agent controller 622 and the regulatory agent controllers 610, including the nominal basal controller 630, the instantaneous basal controller 632, the correction controller 626, and the food-intake controller 628, may be referred to as sub-controllers or as sub-controllers of the controller 202. Moreover, although the controllers of FIG. 6 are illustrated as part of (e.g., as sub-controllers) the controller 202, in some implementations, one or more of the controllers may be separate from the controller 202. In cases where each of the sub-controllers are implemented as separate from the controller 202, the controller 202 itself may be omitted. In other words, each sub-controller may be implemented as an independent controller. Further, some or all of the controllers may be implemented by a processor 530, by the controller 202, or by a combination of the one or more processors 530 and the controller 202.

In the illustrated example, the controller 202 may include four separate controllers. However, it should be understood that more or fewer controllers are possible. In some cases, the controller 202 may be a bihormonal controller capable of controlling the administering of multiple hormones and/or medicaments. For example, some of the sub-controllers of the controller 202 may be regulatory agent controllers 610 while one or more of the controllers may include a counter-regulatory agent controller 622 (e.g., a glucagon controller). The regulatory agent controllers 610 may be configured to prevent or reduce the occurrence or risk of hyperglycemia by causing regulatory agent to be administered by the medicament delivery device 312. The regulatory agent controllers 610 may include a basal controller 624, a correction controller 626, and a food-intake controller 628.

The basal controller 624 may include a controller that can regulate basal insulin delivery. The basal insulin delivery may comprise a regular or periodic delivery of insulin that attempts to maintain a stable or steady glucose level of the subject 512. Basal insulin is often used to maintain the glucose level of the subject 512 outside periods of food-intake or increased activity times (e.g., exercise). Basal insulin may be delivered relatively frequently (e.g., once every five minutes, once an hour, etc.) or relatively less frequently (e.g., once a day, twice a day, etc.). The frequency with which basal insulin is delivered may be based at least in part on the type of insulin delivered. For example, long-acting insulin (LAI) may be delivered less frequently than fast-acting insulin (FAI).

The basal controller 624 may include a nominal basal controller 630 and an instantaneous basal controller 632. The nominal basal controller 630 may control administering of a basal dose over time based on a nominal basal rate. The nominal basal rate may be slowly adapted over time (e.g., on the order of a day). Further, the nominal basal rate may be based on a weight of the subject 512 and/or other subject-specific characteristics. In some cases, irrespective of basal rate adaption, the nominal basal rate may differ during different times of the day. For example, there may be one nominal basal rate during the day and another during the night. As another example, there may be a different nominal basal rate every 6 or 8 hours. Each of these nominal basal rates may be adapted over a longer time period (e.g., on the order of a day) enabling longer-term changes in a subject's insulin needs.

The instantaneous basal controller 632 may adjust the basal dose on a shorter time scale (e.g., every 5-minutes, on the order of an hour, etc.). The instantaneous basal controller 632 may adjust each basal dose at a discrete time interval (e.g., every 5-minutes or every hour). In some cases, the instantaneous basal controller 632 may adjust or adapt the basal dose centered around the nominal basal rate or a nominal basal dose determined based at least in part on the nominal basal rate.

The correction controller 626 may cause regulatory agent (e.g., insulin or insulin analog) to be administered to the subject 512 via the medicament delivery device 312 based at least in part on a glucose level or a change in glucose level of the subject 512. In other words, the correction controller 626 may cause medicament to be delivered to correct a divergence in a desired or target range (e.g., a setpoint range) of the glucose level of the subject 512. This medicament dose may be referred to as a correction dose. In some cases, the delivery of the correction dose may be triggered by the glucose level of the subject 512 exceeding a threshold value (e.g., an upper value of the target range). The correction dose may be selected to modify the glucose level of the subject 512 to be at or within a threshold distance of a setpoint target.

In some cases, the correction controller 626 may implement a model predictive control (MPC) algorithm and may be referred to as a model predictive controller. Alternatively, or in addition, the correction controller 626 may implement a biexponential pharmacokinetic (PK) model as described in some of the Controller Disclosures.

The food-intake controller 628 may cause regulatory agent (e.g., insulin or insulin analog) to be administered to the subject 512 via the medicament delivery device 312 based at least in part on a determination of a food-intake event. In some cases, the food-intake controller 628 may be or may be referred to as a priming insulin controller or a meal controller. The regulatory agent or medicament dose may be referred to as a food-intake dose. The food-intake dose may be a prandial insulin dose, a post-prandial insulin dose, or may be divided into both a prandial and post-prandial insulin dose.

A food-intake event may be associated with the consumption of food and may be determined to occur any time food is consumed. In other cases, a food-intake event may be limited to particular food consumption events, such as meals. Meals may include breakfast, lunch, or dinner, but is not limited to these particular meals. Food-intake events associated with meals may be referred to as meal-intake events. In yet other cases, food-intake events may be associated with the consumption of food of at least a minimum size or quantity of macronutrients. In other words, a snack below a threshold number of calories or carbohydrates may not qualify as a food-intake event while a snack exceeding the threshold number of calories of carbohydrates may quality as a food-intake event, regardless of whether the food-intake event is categorized as a meal.

The food-intake controller 628 may determine whether a food-intake event has occurred, or will occur, based at least in part on a food-intake announcement, which may also be referred to as a meal-announcement. A food-intake announcement may include an interaction by a user with a user interface of the glucose level control system 300 or a device in communication with the glucose level control system 300 that the subject 512 is consuming or will consume a meal. Further, the food-intake announcement may include a qualitative or quantitative indication of a size of the food-intake or of macronutrients of the food-intake. Alternatively, or in addition, the food-intake controller 628 may determine a food-intake event based at least in part on glucose levels of the subject 512.

In some embodiments, food-intake is managed by the correction controller 626. In such embodiments, the correction controller 626 may cause a correction dose of medicament to be administered in response to a food-intake event. Further, in some such embodiments, the food-intake controller 628 may be optional or omitted.

In some embodiments, each of the sub-controllers (e.g., one or more of the regulatory agent controllers 610 and/or the counter-regulatory agent controller 622) of the controller 202 may independently generate an output signal to cause the medicament delivery device 312 to administer medicament. Thus, each sub-controller may provide an output signal to the medicament delivery interface 306. However, as illustrated in FIG. 6, in some cases, two or more of the output signals from two or more of the sub-controllers may be combined to form a medicament (e.g., insulin) dose control signal that is based at least in part on the output of two or more of the two or more sub-controllers. For example, an output signal from the nominal basal controller 630 and an output signal from the instantaneous basal controller 632 may be combined to form a single basal control signal. The basal control signal may be provided to the medicament delivery interface 306, which may cause the medicament delivery device 312 to administer a basal dose of medicament (e.g., insulin or insulin analog) to the subject 512. As another example, the basal control signal may be combined with a correction dose control signal output by the correction controller 626 and/or a food-intake dose control signal output by the food-intake controller 628.

Combining two or more of the control signals may include performing a superposition operation, a merge or join process, and/or a signal aggregation process. Alternatively, or in addition, the controller 202 may receive an output from two or more of the sub-controllers (e.g., the regulatory agent controllers 610 and/or the counter-regulatory agent controller 622) and may generate a signal for output to the medicament delivery interface 306 that is based at least in part on the outputs of each sub-controller. In some cases, the controller 202 may weight the output of each sub-controller equally. In other cases, the controller may weight the output of at least one sub-controller differently from the output of at least one other sub-controller. For example, an output signal from the food-intake controller 628 may be weighted differently than an output signal from the correction controller 626. As another example, an output from the counter-regulatory agent controller 622 may be weighted differently from the output of one or more of the sub-controllers of the regulatory agent controllers 610. In some cases, the output of the counter-regulatory agent controller 622 may be maintained separately from or may supersede an output from the regulatory agent controllers 610.

As mentioned above, the regulatory agent controllers 610 may be configured to prevent or reduce the occurrence or risk of hyperglycemia. Similarly, the counter-regulatory agent controller 622 may be configured to prevent or reduce the occurrence or risk of hypoglycemia. The counter-regulatory agent controller 622 may generate a counter-regulatory agent dose control signal that can be provided to the medicament delivery device 312 to cause the delivery of the counter-regulatory agent (e.g., glucagon) by the medicament delivery device 312. Alternatively, or in addition, the counter-regulatory agent controller 622 may generate a recommendation for a dose of the counter-regulatory agent, which may be output on a display of the glucose level control system 300, an electronic device 252, or a remote computing system 254.

The medicament delivery device 312 may support the delivery of both regulatory and counter-regulatory agent. Alternatively, the medicament delivery device 312 may include two separate delivery devices with one configured to delivery regulatory agent and another configured to delivery counter-regulatory agent. In yet other cases, the medicament delivery device 312 may deliver regulatory agent, and the counter-regulatory agent controller 622 may cause output of a recommendation of counter-regulatory agent when appropriate based at least in part on the glucose level of the subject 512. In yet other embodiments, the medicament delivery device 312 may include two or more delivery devices configured to administer a regulatory agent. For example, the medicament delivery device 312 may include a delivery device configured to administer LAI and not delivery device configured to administer FAI. In yet other cases, the medicament delivery device 312 may be a single delivery device configured to delivery multiple types of regulatory and/or counter-regulatory agents.

The counter-regulatory agent controller 622 may determine to administer a counter-regulatory agent dose or that a counter-regulatory agent dose is recommended based at least in part on a glucose level of the subject 512. In some cases, the counter-regulatory agent dose may be referred to as a correction dose. Thus, in some cases, a correction dose may be a regulatory agent or a counter-regulatory agent. Moreover, in some cases, the operations of the counter-regulatory agent controller 622 may be performed by the correction controller 626 and the counter-regulatory agent controller 622 may be omitted.

As illustrated in FIG. 6, the controller 202 may receive a set of controller inputs 602. The controller inputs 602 may include any inputs that can facilitate the controller 202, or any of the sub-controllers, generating the output signals. For example, the controller inputs 602 may include the one or more parameter inputs 520, the user control inputs 524, a glucose level or glucose level signal received from a glucose level sensor 310, or any other input that can affect operation of the controller 202. Further, the controller 202 may provide some or all of the controller inputs 602 to each of the sub-controllers. In some cases, some of the controller inputs 602 are provided to some of the sub-controllers, while other controller inputs 602 are provided to other sub-controllers of the controller 202. Additionally, as illustrated, in some cases one or more of the sub-controllers may communicate with one or more other sub-controllers of the controller 202. For example, the nominal basal controller 630 may communicate with the instantaneous basal controller 632, which may affect the output of one or more of the nominal basal controller 630 or the instantaneous basal controller 632. As another example, the food-intake controller 628 may communicate with the correction controller 626 and/or the counter-regulatory agent controller 622. Communication between the sub-controllers of the controller 202 may be referred to as inter-controller signals. The inter-controller signals may be used to enable two or more of the sub-controllers to work in conjunction with each other and/or to provide information that may be used for that sub-controller's control function.

The controller 202, or any of its sub-controllers (e.g., the regulatory agent controllers 610 and/or the counter-regulatory agent controller 622 may operate in either an online mode or in an offline mode. Further, the controller 202 or any of its sub-controllers may operate in an automated mode or in a manual mode. In the automated mode, the controller 202 may regulate a glucose level of the subject 512 using one or more control algorithms, such as those disclosed in the Controller Disclosures. Further, the controller 202 may operate using adaptive automated control, such as described in the Controller Disclosures. The control algorithms may use one or more glucose level signals received from the glucose level sensor 310 to determine medicament dosing. The controller 202 may execute control methods or algorithms that include control parameters that are mathematically combined with measured and/or predicted glucose level values to generate an output value that is converted (either directly or via additional conditioning) into one or more dose control signals. For example, the controller 202 may implement a generalized predictive control (GPC) method, as described in U.S. Pat. No. 7,806,854, that incorporates a variety of control parameters. The control algorithms may be generally adaptive. For example, the control parameters of the control algorithms may be dynamically adjusted during operation to reflect changing operating circumstances. Further, by monitoring its own operation, one or more of the control algorithms may implement a “learning” aspect that enables the one or more control algorithms to adjust their operation to be more specifically tailored to an individual subject 512, thereby enhancing the control algorithm's effectiveness and reducing or avoiding a need for additional input information about the subject 512, or other user. It should be noted that the one or more parameter inputs 520, and/or one or more user control inputs 524 may form part of the control parameters used by the control algorithms. Additional control parameters may exist as internal parameters according to the specifics of the control algorithm. Selected control parameters, including one or more parameter inputs 520, user control inputs 524, and/or the internal parameters, may be dynamically adjusted to realize the adaptation of the control algorithm.

In certain embodiments, the controller 202 or the sub-controllers thereof may learn from recent past periods of online operation and may use that learning during offline operation. For example, the Controller Disclosures describe several methods that are usable independently or together to operate in offline mode based at least in part on past operations during online mode. One example method automatically calculates the correct size of a correction bolus of insulin at a time of receiving an isolated glucose measurement based at least in part on past online operation. The correction bolus may be administered by the glucose level control system 300 in response to a user control input 524. A second example method automatically calculates the correct size of a meal bolus (or food-intake bolus) of insulin and administers it in response to a user control input 524. Both methods utilize information obtained during past periods of online operation to automatically calculate correct values, freeing the user of a need to make the calculation or provide a correction factor and improving the accuracy of the medicament dose determination.

It should be understood that the controller 202 or one or more of the sub-controllers of the controller 202 may implement one or more of the algorithms described in the Controller Disclosures. For example, in some cases, the controller 202 and/or one or more of the sub-controllers of the controller 202 may implement a model predictive control (MPC) algorithm as described in some of the Controller Disclosures. Alternatively, or in addition, the controller 202 and/or one or more of the sub-controllers of the controller 202 may implement a biexponential pharmacokinetic (PK) model as described in some of the Controller Disclosures. Further, although each of the sub-controllers included in the controller 202 are described as causing medicament (e.g., insulin, insulin analog, glucagon, etc.) to be administered, in some cases, the controller 202 or its sub-controllers may generate a recommendation of a medicament dose, which may be output on a display for presentation to a user or to another device (e.g., an electronic device 252 or a remote computing system 254).

### Adaptive Glucose Level Control System

In some cases, a user may manage a subject's 512 disease by using the glucose level control system 300. The glucose level control system 300 may be used to control an ambulatory medicament device (e.g., an insulin infusion pump). The ambulatory medicament device may use a control algorithm to help determine a quantity of medicament to deliver to a subject and under what circumstances to deliver the medicament. In some cases, the ambulatory medicament device receives one or more initialization values. The initialization values may be received via user interaction with an interface or retrieved from a memory storage device. In some cases, the glucose level control system 300 sets one or more parameters of a control algorithm based on the initialization values. The glucose level control system 300 may adapt one or more parameters over time based at least in part on initialization values, the subject's 512 past therapy history, and/or glucose levels. For example, the initialization values can serve as a starting point (e.g., initial conditions) for the control algorithm.

Adapting the one or more parameters over time may help improve maintenance of the subject's disease. In some cases, the glucose level control system 300 may adapt the parameters to adjust the medicament deliveries to reduce the error between a subject's 512 predicted glucose level and measured glucose level. For example, the glucose level control system 300 may adjust a basal dose to a modified basal dose to reduce a subject's 512 glycemic excursions. Similarly, the glucose level control system 300 may adapt a total daily dose value or a parameter that corresponds to the total daily dose value. The adapted total daily dose value may be used to determine a medicament therapy. The glucose level control system 300 may also adapt the prediction model. For example, the glucose level control system 300 may change the weight and/or relationships of one or more parameters. Adapting the prediction model may cause the glucose level control system 300 to adapt the subject's 512 medicament therapy more effectively.

In some cases, a subject's 512 glucose level may require different basal rates for different portions of the day. For example, a subject 512 may experience glucose level spikes during particular times of the day. The glucose level control system 300 may use a time-segmented basal dose that may anticipate the periodic fluctuations of the subject's 512 glucose levels. In some cases, the glucose level control system 300 may adapt the time-segmented basal doses to better manage the subject's disease. For example, the adapted time-segmented basal doses may maintain the subject's glucose levels within a target range more effectively.

### Basal Dose Adaptation Process

FIG. 7 presents a flowchart of an example basal dose adaptation process 700 in accordance with certain embodiments. The process 700 may be performed by any system that can adapt a medicament dose (e.g., a basal dose) to be administered to the subject 512 over a time period. For example, the process 700 may be performed by an ambulatory medicament device 250, the glucose level control system 300, and/or an electronic device 252. In some embodiments, the process 700 may be at least partly performed by one or more elements of the glucose level control system 300, such as one or more processors 530, one or more controllers 202, the memory 540, the medicament delivery interface 306, or the interface circuitry 532. In some cases, at least certain operations of the process 700 may be performed by the remote computing system 254 that receives therapy data corresponding to the subject 512. In some embodiments, the therapy data includes one or more basal doses associated with administering medicament to the subject 512. Alternatively, or in addition, the therapy data may include the subject's 512 glucose level data obtained from the glucose level sensor 310 or from isolated glucose measurements 406. The therapy data may correspond to glucose control therapy provided to the subject 512. Although one or more different systems may perform one or more operations of the process 700, to simplify discussions and not to limit the present disclosure, the process 700 is described with respect to particular systems.

The glucose level control system 300 may perform the process 700 to adapt the subject's 512 basal dose so that the subject 512 has improved health outcomes. Improved health outcomes may include reducing the fluctuations of the subject's 512 glucose level and/or increasing the amount of time the subject's 512 glucose level stays within a desired range. In some embodiments, the process 700 may be performed if the subject 512 is switching to a new medicament type (e.g., a different brand of medicament or a medicament with a different time period of action) or if the subject's 512 medicament administration schedule has been modified. For example, the glucose level control system 300 may perform the process 700 if the subject's 512 sleep schedule has changed (e.g., due to a change in employment), which may affect medicament dosing.

In some embodiments, the glucose level control system 300 may performed the process 700 if the subject has recently started using the glucose level control system 300. Alternatively, or in addition, the process 700 may be performed if a subject's 512 malfunctioning glucose level control system 300 has been replaced with a new glucose level control system 300. Alternatively, or in addition, the process 700 may be performed at time-intervals selected by a user. For example, the glucose level control system 300 may perform the process 700 every day, week, or month. In some embodiments, the glucose level control system 300 determines when to perform the process 700 using a control algorithm. For example, the glucose level control system 300 may perform the process 700 when the subject's 512 glucose level leaves a desired range for a certain amount of time. Alternatively, or in addition, the process 700 may be triggered by certain events. In some embodiments, the glucose level control system 300 may perform the process 700 one or more times during a therapy period. For example, the glucose level control system 300 may perform the process 700 when a subject 512 wakes up or goes to sleep.

The process 700 begins at block 702 where, for example, the glucose level control system 300 obtains an indication of a basal dose. The glucose level control system 300 may receive the indication of the basal dose via user interaction with a basal dose entry user interface. For example, the basal dose entry user interface can prompt the user to enter a basal rate and/or a dose size. Alternatively, or in addition, the glucose level control system 300 may receive the indication of the basal dose from the electronic device 252 or the remote computing system 254. The indication of the basal dose may correspond to a particular therapy period. In some embodiments, the therapy period may be a period between two events. For instance, a therapy period may be a period between meals or the period between when the subject 512 wakes up and goes to sleep. Alternatively, or in addition, the therapy period may be a time period of minutes, hours, or days. In some cases, the time period is at least a day.

In some embodiments, the time period corresponding to the therapy period can include a plurality of time segments. For example, a 24-hour time period may be divided into a morning, afternoon, and evening time segment. For instance, the morning time segment may be the first four hours of the time period, the afternoon time segment may be the next eight hours of the time period, and the evening time segment may be the final twelve hours of the time period. In some embodiments, each time segment may have a corresponding basal dose. For example, the morning time segment may have a corresponding basal rate of 1 unit of FAI per hour, the afternoon time segment may have a corresponding basal rate of 0.7 units of FAI per hour, and the evening time segment may have a corresponding basal rate of 0.5 units of FAI per hour. Alternatively, or in addition, each time segment may have a corresponding basal dose that is a single dose. For example, the morning time segment may have a corresponding basal dose of 3 units of LAI, the afternoon time segment may have a corresponding basal dose of 4 units of LAI, and the evening time segment may have a corresponding basal dose of 5 units of LAI.

It should be understood that the time period may include any number of time segments. For example, a time period may include 2, 3, 5, 7, 10, 12, 24, or more time segments, or any number in between. In some embodiments, all the time segments have the same length. For instance, all the time segments may have a length of one hour. Alternatively, or in addition, one or more of the time segments may have different lengths. It should be understood that the time segments may have any length (e.g., 30 minutes, 2 hours, 6 hours, 24 hours, or more, or any length in between). In some embodiments, the time segments may correspond to different events or periods. For example, the time segments may correspond to the subject's 512 meal schedule. For instance, one time segment may correspond to the period between breakfast and lunch, while another time segment may correspond to the period between lunch and dinner.

In some embodiments, the glucose level control system 300 determines a medicament dose based at least partly on the indication of the basal dose received at the block 702. Alternatively, or in addition, the glucose level control system 300 can determine a basal dose based at least in part on historical therapy data (e.g., past basal doses). In some embodiments, the glucose level control system 300 may receive an indication of the basal dose from another computing system, such as the electronic device 252 or the remote computing system 254. The basal dose may be a basal rate (e.g., 1.5 units of FAI per hour) or a single dose (e.g., 5 units of LAI). It should be noted that the basal dose can be FAI, LAI, or any other type of insulin.

In some embodiments at block 702, the glucose level control system 300 may obtain a total daily dose of medicament (e.g., insulin) for the subject 512. Alternatively, or in addition, the glucose level control system 300 may obtain the body weight of the subject 512. The glucose level control system 300 may receive the total daily dose of medicament and/or the body weight of the subject 512 via user interaction with a user interface. For example, the user interface can prompt the user to enter the total daily dose of insulin and/or the body weight of the subject 512. In some embodiments, the glucose level control system 300 can determine the subject's 512 total daily dose of medicament and/or body weight based at least in part on historical therapy data. Alternatively, or in addition, the glucose level control system 300 may receive the subject's 512 total daily dose of medicament and/or body weight from the electronic device 252 or the remote computing system 254.

At block 704, the glucose level control system 300 configures a medicament delivery interface 306 to administer a basal dose of medicament. The basal dose of medicament may be determined based at least in part on the basal dose obtained at the block 702. In some cases, the basal dose of medicament may be equivalent to or may correspond to the indication of the basal dose received at the block 702. For example, if the indication of the basal dose is 13 units, the basal dose of medicament delivered at the block 704 may be 13 units. In other embodiments, the basal dose of medicament may be derived from the indication of the basal dose received at the block 702. For example, if the indication of the basal dose is a basal rate of 0.6 units per hour, the basal dose of medicament may be a function of the basal rate and the number of doses administered within a particular time period. For instance, when basal medicament doses are delivered hourly, the basal dose of medicament may be 0.6 units. But when basal medicament doses are delivered every 5 minutes, the basal medicament dose may be 0.6/20 units, or 0.03 units. And when basal medicament doses are delivered every two hours, the basal medicament dose may be 0.6*2 units, or 1.2 units. It should be understood that other delivery frequencies for basal medicament are possible and that the frequency of basal delivery may depend at least in part on the type of medicament (e.g., FAI vs LAI). The glucose level control system 300 may determine the basal dose of medicament using one or more controllers 202, which may generate a basal control signal based at least in part on the indication of the basal dose received at the block 702. The medicament delivery device 312 may administer the basal dose of medicament in response to the basal controls signal. In some embodiments, the basal dose of medicament may be delivered during or over the particular therapy period associated with the indication of the basal dose obtained at block 702.

At block 706, the glucose level control system 300 determines a predicted glucose level of the subject 512. In some embodiments, the predicted glucose level is based at least in part on a medicament on board value. The medicament on board value may correspond to medicament on board data obtained over the particular therapy period associated with the indication of the basal dose and/or over a therapy period that occurred prior to the particular therapy period. The medicament on board data can include an indication of the insulin on board of the subject 512 (e.g., the amount of insulin that is active or predicted to be active in the subject's 512 body after a medicament bolus or at some time subsequent to a medicament bolus). Alternatively, or in addition, the medicament on board data can include the total amount of insulin on board, the amount of fast-acting insulin on board, the amount of long-acting insulin on board, or the amount of any other type of insulin on board (e.g., rapid-acting insulin, intermediate-acting insulin, ultra-long-acting insulin, etc.). As different subjects may process insulin differently, the determination of the amount of insulin on board for a particular subject may be an estimate that maybe determined based at least in part on total administered insulin, glucose level of the subject, meal announcements, activity level, and any other subject-specific data that may affect the determination of medicament on board.

The medicament on board data may be based at least in part on one or more basal doses and/or one or more non-basal dose (e.g., a food-intake dose, correction bolus, etc.). In some embodiments, the medicament on board data may correspond with the insulin on board of the subject over a therapy period. Alternatively, or in addition, the medicament on board data may include previous determinations of medicament on board values for the subject 512. For example, the medicament on board data may include an indication of one or more medicament on board values of the subject for one or more past therapy periods.

In some embodiments, the predicted glucose level is based at least in part on a food-intake dose or a correction dose. Alternatively, or in addition, the predicted glucose level may be based at least in part on medicament delivery data. The medicament delivery data may be associated with glucose level control agents that have been or may be administered to the subject 512. For example, the medicament delivery data may include a quantity of medicament delivered, a timing of delivery of medicament, a rate of medicament delivery, a type of medicament delivered (e.g., FAI or LAI), or any other type of medicament delivery information that may facilitate determining a state of the subject 512 and/or future medicament delivery. The medicament delivery data may be received via user interaction with a user interface. For example, the glucose level control system 300 may prompt the user via the user interface to enter the medicament delivery data, for example, via the user interface or by causing transmission of the medicament delivery data to the glucose level control system 300. In some embodiments, the electronic device 252 or the remote computing system 254 transmits the medicament delivery data to the glucose level control system 300. Alternatively, or in addition, the medicament delivery data may be received from one or more sensors.

In some embodiments, the medicament delivery data includes a log of past medicament deliveries. Alternatively, or in addition, the log of past medicament deliveries may include the type, size, frequency, concentration of the past medicament deliveries, and/or any other type of medicament delivery data that may be recorded by a tracking or log system. Further, the glucose level control system 300 may track or log past medicament delivery data, or other therapy data (e.g., history of subject glucose levels, meal announcements, etc.). Each entry in the log may include a time stamp indicating when the medicament dose was administered to the subject 512, a glucose level of the subject 512 at the time associated with the time stamp, an indication of a dosing trigger (e.g., a basal dose, a food-intake event, a correction trigger, etc.), or any other information that may be associated with delivery of a medicament dose. The time stamp may include the time, date, and/or therapy period during which the medicament dose was administered. In some embodiments, the medicament delivery data includes a log of previously recommended medicament doses. Further, the medicament delivery data may indicate whether the previously recommended medicament doses were administered to the subject, if a modified dose was administered instead, or if a previously recommended medicament dose was omitted from the subject's treatment. Alternatively, or in addition, the log of previously recommended medicament doses may include the type, size, and/or concentration of the previously recommended and/or administered medicament doses.

In some embodiments, the medicament delivery data may include one or more indications of insulin delivery events (e.g., recommended or administered insulin doses). The insulin delivery events may be long-acting insulin events, fast-acting insulin events, or delivery events corresponding to any other type of insulin. Further, the delivery events may be associated with food-intake, a glycemic excursion, basal, or any other type of delivery event. In some embodiments, the medicament delivery data includes a certain number of indications of past medicament delivery events (e.g., indications of the past 5-20 recommended or administered medicament doses). It should be noted that the medicament delivery data can include indications of medicament delivery events delivered via an ambulatory medicament device, a medicament pen, a patch pump, and/or any other delivery method.

In some embodiments, the predicted glucose level may correspond to a particular therapy period. For example, the predicted glucose level may correspond to the particular therapy period associated with the indication of the basal dose obtained at block 702 or one or more past therapy periods. Alternatively, or in addition, the predicted glucose level may include a plurality of predicted glucose level values. The plurality of predicted glucose level values may be associated with a single administered medicament dose. Each of the predicted glucose level values may be associated with different time points subsequent to the administered medicament dose. For example, the glucose level control system 300 may determine predicted glucose level values for different times during the corresponding therapy period. For instance, the glucose level control system 300 may determine a predicted glucose level value for each five-minute, thirty-minute, or one-hour interval of the corresponding therapy period. It should be understood that the interval may vary based on different treatment plans and/or subject 512 characteristics. Alternatively, or in addition, the plurality of predicted glucose level values may correspond to a plurality of therapy periods. For example, the predicted glucose level may include predicted glucose level values from the previous 2, 5, 10, or more therapy periods, or any number in between.

In some embodiments, the plurality of predicted glucose level values may correspond to one or more medicament on board values. The medicament on board values may correspond to different medicament doses that have been administered to the subject. For example, the plurality of predicted glucose level values may include one or more glucose level values associated with a first medicament on board value and one or more glucose level values associated with a second medicament on board value. The first medicament on board value may correspond to a first administered medicament dose and the second medicament on board value may correspond to a second administered medicament dose. It should be noted that the first and second administered medicament doses may correspond to the same therapy period but may have been administered at different times. The plurality of predicted glucose level values may be based on a medicament on board value that corresponds to multiple administered medicament doses (e.g., the first and second medicament doses).

At block 708, the glucose level control system 300 determines a measured glucose level of the subject 512. The glucose level control system 300 may determine the measured glucose level using the glucose level signal received from the glucose level sensor 310. Alternatively, or in addition, the glucose level control system 300 can determine the measured glucose level using one or more isolated glucose measurements 406.

In some embodiments, the measured glucose level corresponds to a particular therapy period. For example, the measured glucose level may correspond to the particular therapy period associated with the indication of the basal dose obtained at block 702 or one or more past therapy periods. Alternatively, or in addition, the measured glucose level may include a plurality of measured glucose level values. For example, the plurality of measured glucose level values may correspond to different times within the corresponding therapy period. For instance, the glucose level control system 300 may determine a measured glucose level value for each five-minute, thirty-minute, or one-hour interval of the corresponding therapy period. It should be understood that the sampling interval may be any length of time.

In some embodiments, the plurality of measured glucose level values corresponds to a single medicament dose or a plurality of medicament doses. For example, the plurality of measured glucose level values may correspond to the period after a single medicament dose has been administered to the subject 512. Alternatively, or in addition, the plurality of measured glucose level values may correspond to the period after a plurality of medicament doses have been administered to the subject 512. In some embodiments, the measured glucose level may correspond at least in part to one or more food-intake doses and/or one or more correction boluses. For example, the plurality of measured glucose level values may correspond to the period after the subject has received a food-intake dose or a correction bolus.

In some embodiments, the measured glucose level corresponds to one or more qualified time segments of the therapy period. The glucose level control system 300 may qualify a time segment if the time segment satisfies one or more basal evaluation criteria. In some embodiments, the basal evaluation criteria may be used to identify time segments with corresponding glucose levels that are wholly or primarily attributable to the basal dose. Alternatively, or in addition, the glucose level control system 300 may determine time segments with corresponding glucose levels that are attributable to one or more basal doses to at least a threshold degree. By identifying time segments where the effects of food-intake doses and/or correction doses are relatively small compared to the effects of the basal dose, the glucose level control system 300 may be able to adapt the basal dose more effectively.

In some embodiments, a basal evaluation criterion may be satisfied if the glucose level control system 300 determines that the medicament on board value attributable to non-basal medicament deliveries (e.g., food-intake dose, correction bolus, etc.) is less than or equal to a medicament delivery threshold. The medicament delivery threshold may be an amount of medicament and/or a percentage of medicament. For instance, the medicament delivery threshold may be 0 to 5 units of insulin, or more than 5 units of insulin. Alternatively, or in addition, the medicament delivery threshold may be 0% to 60%, or more than 60% of the subject's 512 total amount of insulin on board. For example, the basal evaluation criterion may be satisfied if less than 50% of the subject's 512 total amount of insulin on board corresponds to non-basal medicament deliveries. Similarly, the basal evaluation criterion may be satisfied if the subject's 512 amount of insulin onboard attributable to food-intake doses and correction boluses is less than or equal to 3 units of insulin.

In some embodiments, a basal evaluation criterion is satisfied if the medicament on board value attributable to specific types of non-basal medicament deliveries is less than or equal to the medicament delivery threshold. For example, the basal evaluation criterion may be satisfied if the subject's 512 amount of insulin onboard attributable to one or more food-intake doses is less than or equal to 2 units of insulin. As another non-limiting example, a basal evaluation criterion may be satisfied if the subject's 512 amount of insulin onboard attributable to one or more correction boluses is less than or equal to 2 units of insulin.

In some embodiments, a basal evaluation criterion may be satisfied if the glucose level control system 300 determines that a medicament bolus (e.g., to compensate for food-intake or to compensate for a glucose excursion) beyond a minimum bolus threshold was administered to the subject less than a threshold amount of time prior to the beginning of the time segment. For example, the minimum bolus threshold may be 0 to 15 units of medicament, or more than 15 units of medicament. And the threshold amount of time may be 30 minutes to 5 hours, or more than 5 hours. Thus, in some embodiments, the basal evaluation criterion may be satisfied if the subject 512 has not received a medicament bolus corresponding to food intake of 3 or more units of insulin within 3 hours of the beginning of the time segment. Alternatively, or in addition, the basal evaluation criterion may be satisfied if the subject 512 has not received a medicament bolus corresponding to a glucose excursion of 3 or more units of insulin within 3 hours of the beginning of the time segment.

In some embodiments, a basal evaluation criterion may be satisfied if the glucose level control system 300 determines that the measured glucose level of the subject is within a target range. The target range may vary to accommodate the subject's 512 characteristics. For example, the bottom limit of the target range can be a glucose level of 60 to 90 mg/dL. The upper limit of the target range can be 120 to 200 mg/dL. In some embodiments, the basal evaluation criterion may be satisfied if the measured glucose level is within the target range for the entire time segment. Alternatively, or in addition, the basal evaluation criterion may be satisfied if the measured glucose level is within the target range for a threshold amount of time (e.g., 50% to 100% of the total time) during the time segment.

In some embodiments, a basal evaluation criterion may be satisfied if the glucose level control system 300 determines that the subject is not exercising. In some embodiments, the user may indicate that the subject is exercising via a user interface. Alternatively, or in addition, the glucose level control system 300 may automatically determine that the subject is exercising (e.g., via one or more accelerometers and/or other sensors). In some embodiments, the basal evaluation criterion is satisfied if the subject is not currently exercising or has not exercised within a threshold amount of time (e.g., within the last hour).

In some embodiments, a basal evaluation criterion may be satisfied if the glucose level control system 300 determines that the subject is not experiencing a temporary illness that affects the glucose level of the subject. In some embodiments, the user may indicate that the subject is experiencing an illness via a user interface. Alternatively, or in addition, the glucose level control system 300 may automatically determine that the subject is experiencing an illness (e.g., the glucose level control system 300 determines the that the subject's 512 glucose levels are atypical compared to the subject's glucose level history). The glucose level control system 300 may also receive an indication that the subject is experiencing a temporary illness from an electronic device 252 or a remote computing system 254 (e.g., the computing system of the subject's 512 hospital or doctor).

At block 710, the glucose level control system 300 determines a difference between the predicted glucose level and the measured glucose level of the subject 512. In some embodiments, the glucose level control system 300 determines the difference between the predicted glucose level and the measured glucose level by determining the difference between a plurality of predicted glucose level values and a plurality of measured glucose level values. For example, for each of a set of time periods or points in time, the glucose level control system 300 may determine a difference between a measured glucose level value and a corresponding predicted glucose level value associated with the time period.

In some embodiments, the glucose level control system 300 determines the difference between the predicted and measured glucose levels by determining an average difference between the plurality of predicted glucose level values and the plurality of measured glucose level values. Alternatively, or in addition, the glucose level control system 300 may determine the difference between the predicted and measured glucose levels by determining a trend in the difference of the plurality of predicted glucose level values and the plurality of measured glucose level values. For example, the glucose level control system 300 may be able to determine if the difference between the predicted glucose level and the measured glucose level is increasing or decreasing during the corresponding therapy period. In some embodiments, the glucose level control system 300 determines the difference between the predicted and measured glucose levels by determining the maximum difference between the plurality of predicted glucose level values and the corresponding plurality of measured glucose level values.

At block 712, the glucose level control system 300 adapts the basal dose of medicament based at least in part on the difference between the predicted glucose level and the measured glucose level. In some cases, the glucose level control system 300 may adapt the basal dose based at least in part on the difference between the plurality of predicted glucose level values and the plurality of measured glucose level values. Alternatively, or in addition, the glucose level control system 300 may adapt the basal dose based at least in part on the total daily dose of medicament or the body weight of the subject 512.

In some embodiments, the glucose level control system 300 adapts the basal dose of medicament by modifying the basal dose from a first basal dose to a second basal dose (e.g., the adapted basal dose). The first basal dose may correspond to a user-inputted basal dose obtained at the block 702 and may serve as a starting point for the basal dose adaptation process 700. In some embodiments, the second basal dose is a single medicament dose or a basal rate. It should be noted that the glucose level control system 300 may adapt the basal dose to the second basal dose without user interaction with the basal dose entry user interface.

In some embodiments, the glucose level control system 300 adapts the basal dose so that the subject's disease is better managed. For example, the glucose level control system 300 can adapt the basal dose so that the subject 512 experiences fewer glycemic excursions, hyperglycemic events, and/or hypoglycemic events. Alternatively, or in addition, the glucose level control system 300 can adapt the basal dose if the subject's lifestyle or health condition changes. For example, if the subject 512 gains or loses an amount of weight over a certain threshold (e.g., ±10 kg), the glucose level control system 300 may adapt the basal dose to better manage the subject's 512 glucose level (e.g., reduce fluctuations in the subject's 512 glucose level as compared to the subject's glucose level fluctuations while receiving the first basal dose).

In some embodiments, the difference between the first and second basal doses corresponds to the difference between the predicted and measured glucose levels. For example, the difference between the first and second basal doses increases when the difference between the predicted and measured glucose levels increase. Similarly, the difference between the first and second basal doses may decrease when the difference between the predicted and measured glucose levels decrease. Alternatively, or in addition, the glucose level control system 300 may use a closed-loop control system to adapt the basal dose where the predicted glucose level serves as the setpoint (e.g., the desired glucose level), and the measured glucose level serves as one of the process variables (e.g., the measured value in the feedback loop).

In some embodiments, the glucose level control system 300 may adapt the type, size, frequency, and/or concentration of the basal dose. For example, if the glucose level control system 300 determines that the subject 512 needs more medicament, the glucose level control system 300 may adapt the basal dose by increasing the basal rate (e.g., from 1 unit of FAI per hour to 1.5 units of FAI per hour). Similarly, if the glucose level control system 300 determines that the subject 512 needs less medicament, the glucose level control system 300 may adapt the basal dose by decreasing the basal rate (e.g., from 1 unit of FAI per hour to 0.5 units of FAI per hour). Alternatively, or in addition, the glucose level control system 300 may adapt the basal dose by increasing or decreasing the size of a single dose of medicament (e.g., from 4 units of LAI to 5 units of LAI).

In some embodiments, the glucose level control system 300 adapts the basal dose for a corresponding therapy period. The basal dose may be a single medicament dose or a basal rate. In some embodiments, the basal rate may be a nominal basal rate (e.g., 1.5 units of medicament per hour) or an instantaneous basal rate.

The glucose level control system 300 may use the first basal dose for a first therapy period, and adapt the basal dose to the second basal dose for a second therapy period. Thus, the first basal dose may correspond to the medicament administered over a first therapy period, and the second basal dose may correspond to the medicament administered over a second therapy period. In some embodiments, the first basal dose is the medicament dose associated with the indication of the basal dose obtained at block 702, and the first therapy period is the particular therapy period associated with the indication of the basal dose at block 702. In some embodiments, the glucose level control system 300 may determine that the first basal dose does not need to be adapted or that the recommended basal dose matches the first basal dose. Thus, in some embodiments, the first and second basal doses may be the same.

In some embodiments, the glucose level control system 300 adapts the basal dose for each time segment of the corresponding therapy period. For example, if the corresponding therapy period has three time segments, the glucose level control system 300 may adapt the basal doses corresponding to each time segment, which may result in different adapted basal doses for each time segment, or at least two of the time segments may have the same adapted basal dose. In some embodiments, the glucose level control system 300 may determine that one or more of the first basal doses do not need to be adapted. Thus, in some embodiments, one or more of the corresponding first and second basal doses may be the same.

In some embodiments, the glucose level control system 300 may repeat the process 700 for additional therapy periods. For example, the glucose level control system 300 may adapt the second basal dose to a third basal dose that corresponds to medicament administered over a third therapy period. In some embodiments, the glucose level control system 300 repeats the process 700 with therapy periods that include receding time horizons. For example, the therapy period may be a sliding window (e.g., the therapy period may include data from the most recent day, week, or preceding time period). Thus, the glucose level control system 300 may be able to repeat the process 700 in shorter intervals than the length of the therapy period. For example, the glucose level control system 300 may repeat the process 700 every hour using day-long therapy periods (e.g., the second therapy period is shifted one hour forward compared to the first therapy period). The process 700 may automatically adapt a basal rate so that the subject 512 has better health outcomes. For example, the process 700 may reduce the fluctuations of the subject's 512 glucose level and/or reduce the subject's 512 risk of experiencing hypoglycemic events.

### Time-Segmented Basal Dose Adaptation Process

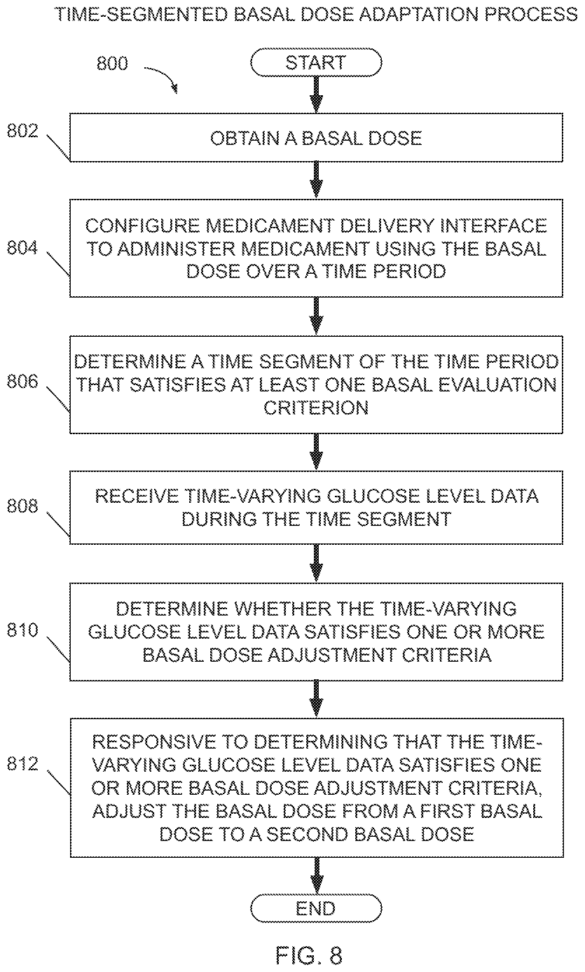

FIG. 8 presents a flowchart of an example time-segmented basal dose adaptation process 800 in accordance with certain embodiments. The process 800 may be performed by any system that can adapt a time-segmented medicament dose (e.g., a basal dose) to be administered to the subject 512 over a time period. For example, the process 800 may be performed by an ambulatory medicament device 250, the glucose level control system 300, and/or an electronic device 252. In some embodiments, the process 800 may be at least partly performed by one or more elements of the glucose level control system 300, such as one or more processors 530, one or more controllers 202, the memory 540, the medicament delivery interface 306, or the interface circuitry 532. In some cases, at least certain operations of the process 800 may be performed by the remote computing system 254 that receives therapy data corresponding to the subject 512. Although one or more different systems may perform one or more operations of the process 800, to simplify discussions and not to limit the present disclosure, the process 800 is described with respect to particular systems.

The glucose level control system 300 may perform the process 800 for the same events, circumstances, and time-intervals as the process 700. For example, the process 800 may be performed if the subject 512 is switching to a new medicament type, if the subject's 512 medicament administration schedule has been modified, if the subject has recently started using the glucose level control system 300, etc.

The process 800 begins at block 802 where, for example, the glucose level control system 300 obtains an indication of a basal dose. The glucose level control system 300 may receive the indication of the basal dose via user interaction with a basal dose entry user interface. The indication of the basal dose may correspond to a particular therapy period. Alternatively, or in addition, the therapy period may be a time period of minutes, hours, or days. In some cases, the time period is at least a day. In some embodiments, the time period corresponding to the therapy period can include a plurality of time segments. The glucose level control system 300 may determine a medicament dose based at least partly on the indication of the basal dose received at the block 802. In some embodiments at block 802, the glucose level control system 300 may obtain a total daily dose of medicament (e.g., insulin) for the subject 512 and/or the body weight of the subject 512. In some embodiments, the block 802 may include one or more of the embodiments previously described with respect to the block 702.

At block 804, the glucose level control system 300 configures a medicament delivery interface 306 to administer a basal dose of medicament. The basal dose of medicament may be determined based at least in part on the basal dose obtained at the block 802. In some cases, the basal dose of medicament may be equivalent to or may correspond to the indication of the basal dose received at the block 802. The glucose level control system 300 may determine the basal dose of medicament using one or more controllers 202, which may generate a basal control signal based at least in part on the indication of the basal dose received at the block 802. The medicament delivery device 312 may administer the basal dose of medicament in response to the basal controls signal. In some embodiments, the basal dose of medicament may be delivered during or over the particular therapy period associated with the indication of the basal dose obtained at block 802. In some embodiments, the block 804 may include one or more of the embodiments previously described with respect to the block 704.

At block 806, the glucose level control system 300 determines a time segment of a time period that satisfies at least one basal evaluation criterion. The time period may correspond to the particular therapy period associated with the indication of the basal dose obtained at block 802. Alternatively, or in addition, the glucose level control system 300 may determine a plurality of time segments of the time period that each satisfy at least one basal evaluation criterion. In some embodiments, the glucose level control system 300 qualifies the time segments that satisfy at least one basal evaluation criterion. In some embodiments, the basal evaluation criteria of block 806 may include one or more of the basal evaluation criteria previously described with respect to the block 708.

At block 808, the glucose level control system 300 receives time-varying glucose level data of the subject 512 during the one or more qualified time segments. In some embodiments, the time-varying glucose level data may include a plurality of glucose level values. The glucose level control system 300 may receive the time-varying glucose level data via user interaction with a user interface. Alternatively, or in addition, the glucose level control system 300 may receive the time-varying glucose level data from the electronic device 252 or the remote computing system 254.

The time-varying glucose level data may be predicted and/or measured time-varying glucose level data. In some embodiments, the predicted time-varying glucose level data is based at least in part on one or more medicament on board values, food-intake doses, and/or correction doses. Alternatively, or in addition, the predicted time-varying glucose level data may be based at least in part on medicament delivery data. In some embodiments, the glucose level control system 300 receives the measured time-varying glucose level data from the glucose level sensor 310 or from one or more isolated glucose measurements 406. In some embodiments, the block 808 may include one or more of the embodiments previously described with respect to the blocks 706 and/or 708.

At block 810, the glucose level control system 300 determines whether the time-varying glucose level data corresponding to the qualified time segments satisfies at least one basal dose adjustment criterion. In some embodiments, a basal dose adjustment criterion may be satisfied if the glucose level control system 300 determines that the percentage of time that the time-varying glucose level data is within a target range is below a threshold percentage. The target range may vary to accommodate the subject's 512 characteristics. For example, the bottom limit of the target range can be a glucose level of 60 to 90 mg/dL. The upper limit of the target range can be 120 to 200 mg/dL. The threshold percentage can be any percentage. For example, the threshold percentage may be 5%, 20%, 40%, 90%, more than 90%, or any percentage in between. For instance, the basal dose adjustment criterion may be satisfied if the time-varying glucose level data is within the target range for less than 80% of the time corresponding to a qualified time segment.

If the glucose level control system 300 has qualified a plurality of time segments, the percentage of time that the time-varying glucose level data is within a target range may be determined for each qualified time segment individually or for all the time segments combined. For example, the basal dose adjustment criterion may be satisfied if the time-varying glucose level data corresponding to all the qualified time segments is within the target range for less than 60% of the time corresponding to all the qualified time segments. Alternatively, or in addition, the basal dose adjustment criterion may be satisfied if the time-varying glucose level data of at least one of the qualified time segments is within the target range for less than 60% of the time corresponding to the same qualified time segment.

In some embodiments, the basal dose adjustment criterion may be satisfied if a threshold percentage of the qualified time segments individually satisfy the requirements of the basal dose adjustment criterion. For example, the basal dose adjustment criterion may be satisfied if at least 60% of the qualified time segments individually satisfy the requirements of the basal dose adjustment criterion. The threshold percentage of qualified time segments may vary from 5%, 10%, 30%, 50%, 100%, or any percentage in between. Alternatively, or in addition, the basal dose adjustment criterion may be satisfied if a threshold number of qualified time segments of the plurality of qualified time segments individually satisfy the requirements of the basal dose adjustment criterion. The threshold number of qualified time segments may vary from 1, 2, 5, 10, or more than 10 qualified time segments or any number in between.

In some embodiments, the glucose level control system 300 may determine whether the average of the time-varying glucose level data satisfies the basal dose adjustment criterion. For example, the basal dose adjustment criterion may be satisfied if the average of the time-varying glucose level data corresponding to all the qualified time segments is within the target range for less than 70% of the time corresponding to the qualified time segments. Alternatively, or in addition, the glucose level control system 300 may determine whether the median of the time-varying glucose level data satisfies the basal dose adjustment criterion. For example, the basal dose adjustment criterion may be satisfied if the median of the time-varying glucose level data corresponding to all the qualified time segments is within the target range for less than 70% of the time corresponding to the qualified time segments.

In some embodiments, a basal dose adjustment criterion may be satisfied if the glucose level control system 300 determines that the time-varying glucose level data is outside the target range at the conclusion of a corresponding qualified time segment. If the glucose level control system 300 has qualified a plurality of time segments, the basal dose adjustment criterion may be satisfied if the time-varying glucose level data is outside the target range at the conclusion of a threshold percentage or threshold number of qualified time segments. For example, the basal dose adjustment criterion may be satisfied if the time-varying glucose level data is outside the target range at the conclusion of at least 50% of the qualified time segments. As described above, the threshold percentage or threshold number of qualified time segments may be any percentage or number of qualified time segments. Alternatively, or in addition, the basal dose adjustment criterion may be satisfied if the average of the last (e.g., concluding) glucose level values of the time-varying glucose level data is outside the target range. Similarly, the basal dose adjustment criterion may be satisfied if the median of the last glucose level values of the time-varying glucose level data is outside the target range.

In some embodiments, a basal dose adjustment criterion may be satisfied if the glucose level control system 300 determines that the time-varying glucose level data is outside the target range for at least a portion of a qualified time segment. For example, the basal dose adjustment criterion may be satisfied if the time-varying glucose level data is outside the target range for a certain amount of time. The amount of time of the portion may vary. For example, the amount of time may be 5 minutes, 30 minutes, 1 hour, 6 hours, 12 hours, or more than 12 hours, or any amount of time in between. For instance, the basal dose adjustment criterion may be satisfied if the time-varying glucose level data is outside the target range for at least a two-hour portion of the qualified time segment. If the glucose level control system 300 has qualified a plurality of time segments, the basal dose adjustment criterion may be satisfied if the time-varying glucose level data is outside the target range for a portion of at least one of the time segments of the plurality of time segments. For example, if the portion corresponding to the basal dose adjustment criterion is a thirty-minute portion, the basal dose adjustment criterion may be satisfied if the time-varying glucose level data is outside of the target range for thirty minutes of at least one of the qualified time segments. Alternatively, or in addition, the basal dose adjustment criterion may be satisfied if the time-varying glucose level data is outside the target range for a combined portion across all the time segments. For example, if the portion corresponding to the basal dose adjustment criterion is a one-hour portion, the basal dose adjustment criterion may be satisfied if the time-varying glucose level data is outside the target range for forty minutes during a first qualified time segment and forty minutes during a second qualified time segment. Alternatively, or in addition, the basal dose adjustment criterion may be satisfied if the average or median of the time-varying glucose level data is outside the target range for a portion of the qualified time segments.

In some embodiments, a basal dose adjustment criterion may be satisfied if the glucose level control system 300 determines that the time-varying glucose level data differs from the target range by more than a threshold difference for at least a portion of a qualified time segment. The threshold difference may be ±1 mg/dL, 10 mg/dL, 15 mg/dL, 20 mg/dL, 40 mg/dL, more than 40 mg/dL, or any number in between. For example, the basal dose adjustment criterion may be satisfied if the time-varying glucose level data is 20 mg/dL above the upper limit of the target range for at least thirty minutes of the corresponding time segment. As another example, the basal dose adjustment criterion may be satisfied if the time-varying glucose level data is 20 mg/dL below the lower limit of the target range for at least thirty minutes of the corresponding time segment. In some embodiments, the portion is a percentage of the time segment. It should be understood that the required portion to satisfy the basal dose adjustment criterion may be any percentage of the corresponding time segment. For instance, the required portion may be 1%, 10%, 20%, 50%, more than 50% or any number in between. For example, the basal dose adjustment criterion may be satisfied if the time-varying glucose level data is 10 mg/dL or more below the lower limit of the target range for at least 20% of the corresponding time segment. If the glucose level control system 300 has qualified a plurality of time segments, the basal dose adjustment criterion may be satisfied if the time-varying glucose level data differs from the target range by more than the threshold difference for the required portion during at least one of the time segments of the plurality of time segments. Alternatively, or in addition, the basal dose adjustment criterion may be satisfied if the time-varying glucose level data differs from the target range by more than the threshold difference for the required portion across all the time segments combined. Additionally, as described above, the glucose level control system 300 may use the average or median of the time-varying glucose level data to determine whether the basal dose adjustment criterion has been satisfied.

In some embodiments, a basal dose adjustment criterion may be satisfied if the glucose level control system 300 determines that the rate of change (e.g., slope) of the time-varying glucose level data corresponding to a qualified time segment satisfies a rate of change threshold (e.g., slope threshold). For example, if the threshold rate of change is a glucose level increase of 60 mg/dL per hour, the basal dose adjustment criterion may be satisfied if the time-varying glucose level data increases more than 60 mg/dL per hour. Similarly, if the threshold rate of change is a glucose level decrease of 60 mg/dL per hour, the basal dose adjustment criterion may be satisfied if the time-varying glucose level data decreases more than 60 mg/dL per hour. It should be noted that the threshold rate of change may vary based on different subject 512 characteristics. For example, the threshold rate of change may be a glucose level increase and/or decrease of 10 mg/dL, 60 mg/dL, 90 mg/dL, 120 mg/dL, or greater than 120 mg/dL, or any amount in between, during a certain time period. The time period over which the rate of change is evaluated may also vary. For example, the time period may be 30 minutes, 1 hour, 2 hours, 4 hours, 8 hours, or more than 8 hours. In some embodiments, the glucose level control system 300 may have a different rate of change threshold for a glucose level increase than a glucose level decrease. For example, the threshold rate of change for the time-varying glucose level data may be an increase of 100 mg/dL per hour and a decrease of 50 mg/dL per hour. If the glucose level control system 300 has determined a plurality of qualified time segments, the average rate of change of the qualified time segments may be compared against the rate of change threshold. Alternatively, or in addition, the median rate of change of the qualified time segments may be compared against the rate of change threshold to determine whether the time segments have satisfied the basal dose adjustment criterion.

In some embodiments, a basal dose adjustment criterion may be satisfied if the glucose level control system 300 determines that the rate of change of a trendline fit corresponding to the time-varying glucose level data satisfies a rate of change threshold. The rate of change threshold may vary as described above. In some embodiments, the glucose level control system 300 determines the rate of change of a linear trendline corresponding to the time-varying glucose level data. Alternatively, or in addition, the glucose level control system 300 may determine the rate of change of a polynomial, exponential, logarithmic, or power trendline corresponding to the time-varying glucose level data. In some embodiments, if the threshold rate of change is an increase of 50 mg/dL per hour, the basal dose adjustment criterion may be satisfied if the rate of change of the trendline fit corresponding to the time-varying glucose level data increases more than 50 mg/dL per hour. Similarly, if the threshold rate of change is a decrease of 60 mg/dL per hour, the basal dose adjustment criterion may be satisfied if the rate of change of the trendline fit corresponding to the time-varying glucose level data decreases more than 60 mg/dL per hour. If the trendline fit has multiple rates of change, the glucose level control system 300 may compare the maximum, minimum, or average rate of change to the rate of change threshold. If the glucose level control system 300 has determined a plurality of qualified time segments, the average rate of change of the trendline fit may be compared against the rate of change threshold to determine whether the time segments have satisfied the basal dose adjustment criterion. Alternatively, or in addition, the median rate of change of the trendline fit may be compared against the rate of change threshold to determine whether the time segments have satisfied the basal dose adjustment criterion.

At block 812, responsive to determining that the time-varying glucose level data of the one or more time segments satisfies one or more basal dose adjustment criteria, the glucose level control system 300 may adjust the basal dose from a first basal dose to a second basal dose. The first basal dose may correspond to medicament administered over a first therapy period, and the second basal dose may correspond to medicament administered over a second therapy period. The second therapy period may have a plurality of time segments. In some embodiments, the first basal dose may correspond to the indication of the basal dose obtained at the block 802 and may serve as a starting point for the time-segmented basal dose adaptation process 800. Alternatively, or in addition, the first therapy period may be the particular therapy period associated with the indication of the basal dose obtained at block 802. It should be understood that the first and second basal doses may be single doses or basal rates. In some embodiments, the block 812 may include one or more of the embodiments previously described with respect to the block 712.

In some embodiments, the glucose level control system 300 may adapt the basal dose based at least in part on the basal dose adjustment criteria that are satisfied by the corresponding time segments. The glucose level control system 300 may determine that the subject's 512 glucose level is high or low based at least in part on the basal dose adjustment criteria that are satisfied. For example, the glucose level control system 300 may determine that the subject's glucose level is high if the satisfied basal dose adjustment criterion corresponds to the time-varying glucose level data being above a target range. Alternatively, or in addition, the glucose level control system 300 may determine that the subject's 512 glucose level is high if the satisfied basal dose adjustment criterion corresponds to the time-varying glucose level data having a rate of change that is greater than a rate of change threshold. In some embodiments, the glucose level control system 300 may determine that the subject's 512 glucose level is high if the subject's 512 glucose level is near the upper limit of a target range (e.g., in the top 5%, 10%, or 20% of the target range) or above the upper limit of the target range.

In some embodiments, the glucose level control system 300 may determine that the subject's 512 glucose level is low if the satisfied basal dose adjustment criterion corresponds to the time-varying glucose level data being below a target range. Alternatively, or in addition, the glucose level control system 300 may determine that the subject's 512 glucose level is low if the satisfied basal dose adjustment criterion corresponds to the time-varying glucose level data having a rate of change that is less than a rate of change threshold. In some embodiments, the glucose level control system 300 may determine that the subject's 512 glucose level is low if the subject's 512 glucose level is near the lower limit of the target range (e.g., in the bottom 5%, 10%, or 20% of the target range) or below the lower limit of the target range. In some embodiments, the target range corresponds to a range of healthy glucose level values for the subject 512 (e.g., the range where the risk of hyperglycemic and/or hypoglycemic events are reduced).

In some embodiments, the glucose level control system 300 sets the second basal dose to be larger than the first basal dose if the glucose level control system 300 determines that the glucose level of the subject is high. For example, the glucose level control system 300 may adjust the basal dose from a first basal rate of 2 units of FAI per hour to a second basal rate of 2.75 units of FAI per hour. As another example, the glucose level control system 300 may adjust the basal dose from a first basal dose of 5 units of LAI to a second basal dose of 8 units of LAI. Alternatively, or in addition, the glucose level control system 300 may set the second basal dose to be smaller than the first basal dose if the glucose level control system 300 determines that the glucose level of the subject is low. For example, the glucose level control system 300 may adjust the basal dose from a first basal rate of 4 units of FAI per hour to a second basal rate of 3 units of FAI per hour. As another example, the glucose level control system 300 may adjust the basal dose from a first basal dose of 6 units of LAI to a second basal dose of 5.5 units of LAI.

In some embodiments, the glucose level control system 300 may adapt the basal dose based at least in part on the time-varying glucose level data of the subject 512 during a set of time segments from the plurality of time segments. The glucose level control system 300 may adapt the basal dose at least partly based on the time-varying glucose level data corresponding to a set of time segments that satisfied a particular basal dose adjustment criterion. For instance, the second basal dose may at least be partly based on the time-varying glucose level data corresponding to the set of time segments where the subject's 512 glucose level was outside a target range for a percentage of time or for a portion of the time segments. In some embodiments, the second basal dose may be increased if the time-varying glucose level data corresponding to the set of time segments is mostly above the target range. Similarly, the second basal dose may be decreased if the time-varying glucose level data is mostly below the target range. If a portion of the time-varying glucose level data is above the target range, and a portion of the time-varying glucose level data is below the target range, the second basal dose may be the same as the first basal dose. Alternatively, or in addition, the glucose level control system 300 may adapt the basal dose based at least in part on the average of the time-varying glucose level data corresponding to the set of time segments. For instance, the second basal dose may be increased if the average time-varying glucose level data corresponding to the set of time segments is mostly above the target range and decreased if the average of the time-varying glucose level data is mostly below the target range.

Similarly, the second basal dose may at least be partly based on the time-varying glucose level data corresponding to the set of time segments where the rate of change satisfied a rate of change threshold. For example, the second basal dose may be increased if the time-varying glucose level data corresponding to the set of time segments has a rate of change that is mostly greater than a rate of change threshold (e.g., an upper rate of change threshold). Similarly, the second basal dose may be decreased if the time-varying glucose level data has a rate of change that is mostly less than a rate of change threshold (e.g., a lower rate of change threshold). If a portion of the time-varying glucose level data has a rate of change that is above a rate of change threshold, and a portion of the time-varying glucose level data has a rate of change that is below a rate of change threshold, the second basal dose may be the same as the first basal dose. Alternatively, or in addition, the glucose level control system 300 may adapt the basal dose based at least in part on the average rate of change of the time-varying glucose level data corresponding to the set of time segments.

In some embodiments, the difference between the first and second basal doses corresponds to the one or more characteristics of time-varying glucose level data. For example, the difference between the first and second basal dose may increase as the percentage of time the time-varying glucose level data was outside a target range increases. Similarly, the difference between the first and second basal dose may increase as the difference between the rate of change of the time-varying glucose level data and the rate of change threshold increases. It should be understood that the glucose level control system 300 may adapt the basal dose based on a variety of difference characteristics and properties of the time-varying glucose level data.

In some embodiments, the glucose level control system 300 adapts the basal dose for each time segment of the corresponding therapy period. For example, if the corresponding therapy periods have five time segments, the glucose level control system 300 may adapt five first basal doses corresponding to the first therapy period to five second basal doses corresponding to the second therapy period. It should be understood the some or all of the time segments may have the same basal doses. For example, the first and second time segments of the first therapy period may have the same first basal dose. Alternatively, or in addition, each time segment corresponding to the first therapy period may have a different corresponding first basal dose. For example, the first, second, and third time segments of the first therapy period may have different first basal doses. In some embodiments, the time segments may correspond to a particular time period within a day. For example, a first time segment may correspond to the morning period of the day (e.g., 8:00-12:00), the second time segment may correspond to the afternoon period of the day (e.g., 12:00-18:00), the third time segment may correspond to the evening period of the day (18:00-21:00), etc. In some embodiments, the glucose level control system 300 may adapt the basal dose to be the same for two or more time segments of the second therapy period. For example, the first and second time segments of the second therapy period may have the same second basal dose. Alternatively, or in addition, the glucose level control system 300 may adapt the basal dose to be different for two or more time segments of the second therapy period. For example, the second and third time segments of the second therapy period may have different second basal doses. In some embodiments, the glucose level control system 300 may determine that one or more of the first basal doses do not need to be changed. Thus, in some embodiments, one or more of the corresponding first and second basal doses may be the same. The multiple basal rates for the different time segments may allow the subject's medicament therapy to be time-segmented. For example, if the subject 512 experiences a rise of blood sugar in the morning period, the glucose level control system 300 may adjust the basal dose corresponding to the morning time segment to be larger. Thus, the time-segmented therapy may maintain the subject's 512 glucose level within a desired range more effectively.

In some embodiments, the glucose level control system 300 may repeat the process 800 for additional therapy periods. For example, the glucose level control system 300 may adapt the second basal dose to a third basal dose that corresponds to medicament administered over a third therapy period. The third therapy period may include a plurality of time segments. In some embodiments, the glucose level control system 300 repeats the process 800 with therapy periods that include receding time horizons. For example, the therapy period may be a sliding window (e.g., the therapy period may include data from the most recent day, week, or preceding time period). Thus, the glucose level control system 300 may be able to repeat the process 800 in shorter intervals than the length of the therapy period. For example, the glucose level control system 300 may repeat the process 800 every hour using day-long therapy periods (e.g., the second therapy period is shifted one hour forward compared to the first therapy period).

### Prediction Model Adaptation Process

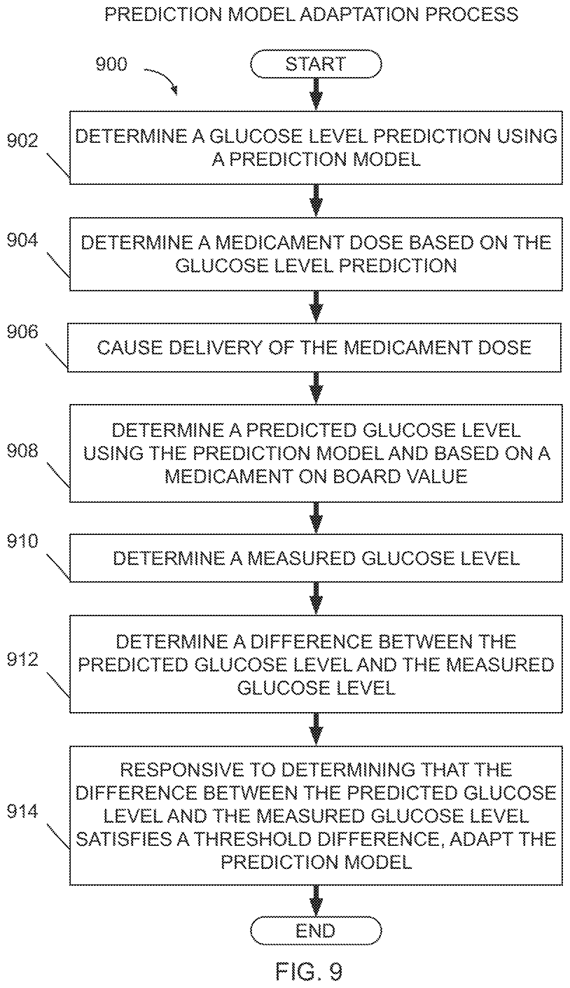

FIG. 9 presents a flowchart of an example prediction model adaptation process 900 in accordance with certain embodiments. The process 900 may be performed by any system that can adapt a prediction model that may be used to determine medicament doses to be administered to the subject 512. For example, the process 900 may be performed by an ambulatory medicament device 250, the glucose level control system 300, and/or an electronic device 252. In some embodiments, the process 900 may be at least partly performed by one or more elements of the glucose level control system 300, such as one or more processors 530, one or more controllers 202, the memory 540, the medicament delivery interface 306, or the interface circuitry 532. In some cases, at least certain operations of the process 900 may be performed by the remote computing system 254 that receives therapy data corresponding to the subject 512. In some embodiments, the therapy data includes one or more basal doses associated with administering medicament to the subject 512. Alternatively, or in addition, the therapy data may include the subject's 512 glucose level data obtained from the glucose level sensor 310 or from isolated glucose measurements 406. The therapy data may correspond to glucose control therapy provided to the subject 512. Although one or more different systems may perform one or more operations of the process 900, to simplify discussions and not to limit the present disclosure, the process 900 is described with respect to particular systems.

The glucose level control system 300 may perform the process 900 for the same events, circumstances, and time-intervals as the process 700 and the process 800. For example, the process 900 may be performed if the subject 512 is switching to a new medicament type, if the subject's 512 medicament administration schedule has been modified, if the subject has recently started using the glucose level control system 300, etc.

The process 900 begins at block 902 where, for example, the glucose level control system 300 determines a glucose level prediction using a prediction model. In some embodiments, the glucose level prediction may include a plurality of predicted glucose levels. The prediction model may use a control algorithm to determine the glucose level prediction. In some embodiments, the glucose level prediction may be based at least in part on one or more medicament on board values, food-intake doses, and/or corrections doses. The medicament on board value may be based at least in part on one or more non-basal doses (e.g., food-intake doses and/or correction doses). In some embodiments, the glucose level prediction may correspond to one or more therapy periods. Alternatively, or in addition, the glucose level prediction may be based at least in part on medicament delivery data and/or a total daily dose value.

In some embodiments, the glucose level prediction is based at least in part on one or more parameters, or values of the parameters, of the prediction model. For example, the glucose level prediction may be based at least in part on a target setpoint parameter. In some embodiments, the target setpoint parameter may be a glucose level target or a medicament (e.g., insulin) level target. The glucose level target may be a glucose level value or a glucose level range. In some embodiments, the glucose level prediction may be based at least in part on a subject model parameter. The subject model parameter may be based at least in part on the weight, age, sex, gender, puberty status, race, and/or ethnicity the subject 512. In some embodiments, the glucose level prediction may be based at least in part on a glucose level prediction parameter. The glucose level prediction parameter may be based at least in part on the subject's 512 metabolism, dietary habits, biorhythm (e.g., sleep cycle), exercise, stress, and/or sleep.

In some embodiments, the glucose level prediction may be based at least in part on a dosing aggressiveness parameter. The dosing aggressiveness parameter may be based at least in part on the operating mode of the glucose level control system 300. For example, the dosing aggressiveness parameter may differ based on whether the glucose level control system 300 is configured to treat a type 1 diabetic or a type 2 diabetic. In some embodiments, the glucose level prediction may be based at least in part on a dosing setting parameter. The dosing setting parameter may be based on the concentration, type, and/or size of the insulin the subject 512 has received or is scheduled to receive. Alternatively, or in addition, the dosing setting parameter may be based on the capacity of the medicament cartridge being used to administer medicament to the subject 512. In some embodiments, the dosing setting parameter is based on a meal announcement dose size, food intake size, carbohydrate ratio, correction dose size, correction factor, and/or basal rate corresponding to the subject 512.

In some embodiments, the glucose level prediction may be based at least in part on an insulin pharmacokinetic parameter. The insulin pharmacokinetic parameter may be based on the Tₘₐₓ and/or T_{1/2max}. Tₘₐₓ may correspond to the time at which the concentration of insulin in the subject's 512 blood reaches a maximum level, and T_{1/2max} may correspond to the time when the concentration of insulin in the blood plasma reaches half of the maximum concentration. Alternatively, or in addition, the insulin pharmacokinetic parameter may be based on an extinction coefficient, time of action of insulin, duration of action of insulin, and/or insulin absorption rate. In some embodiments, the glucose level prediction may be based at least in part on an insulin sensitivity parameter. In some embodiments, the insulin sensitivity parameter may be based at least in part on how quickly and/or how much a subject's 512 glucose level responds to a particular dose of insulin. In some embodiments, the block 902 may include one or more of the embodiments previously described with respect to the blocks 706 and/or 808.

At block 904, the glucose level control system 300 determines a medicament dose based on the glucose level prediction. In some embodiments, the glucose level control system 300 uses a control algorithm to determine the medicament dose. In some embodiments, the glucose level control system 300 may determine a larger medicament dose if the glucose level prediction is high (e.g., on the upper end of a target range) as compared to a medicament dose determined when the glucose level prediction was low (e.g., on the lower end of the target range). Alternatively, or in addition, the glucose level control system 300 may determine a smaller medicament dose if the glucose level prediction is low as compared to a medicament dose determined when the glucose level prediction was high. In some embodiments, the glucose level control system 300 may determine multiple medicament doses based on the glucose level prediction. The glucose level control system 300 may use the glucose level prediction to determine the frequency, concentration, and type of medicament corresponding to the one or more medicament doses. It should be understood that the one or more medicament doses may be basal doses (e.g., single dose or basal rate), food-intake doses, and/or a correction bolus. In some embodiments, the block 904 may include one or more of the embodiments previously described with respect to the blocks 704, 804, and/or 812.

At block 906, the glucose level control system 300 causes a medicament dose to be administered to the subject 512. In some embodiments, the glucose level control system 300 generates a dose control signal based on the medicament dose determined at block 904. The medicament delivery device 312 may administer the medicament dose to the subject 512 in response to the basal controls signal. In some embodiments, the medicament dose may be administered to a subject 512 during a particular therapy period. The therapy period may be a time period. The time period may be a period of hours, days, or weeks. For example, the time period may be a 24-hour period. In some embodiments, the block 906 may include one or more of the embodiments previously described with respect to the blocks 702, 704, and/or 804.

At block 908, the glucose level control system 300 determines a predicted glucose level using the prediction model and based at least in part on a medicament on board value. In some embodiments, the glucose level control system 300 may determine a plurality of predicted glucose levels. The plurality of predicted glucose levels may correspond to a single medicament dose or multiple medicament doses. For example, the plurality of predicted glucose levels may correspond to the one or more medicament doses administered at block 906. In some embodiments, the predicted glucose level may correspond to a therapy period. For example, the corresponding therapy period may be the particular therapy period associated with block 906. As described above, the medicament on board value may be based at least in part on one or more non-basal doses. Alternatively, or in addition, the medicament on board value may be based at least in part on the medicament dose corresponding to block 906. In some embodiments, the predicted glucose level may decrease if the medicament on board value increases. Similarly, the predicted glucose level may increase if the medicament on board value decreases. In some embodiments, the block 906 may include one or more of the embodiments previously described with respect to the blocks 706, 808, and 902.

At block 910, the glucose level control system 300 may determine a measured glucose level. In some embodiments, the measured glucose level may correspond to a therapy period. For example, the corresponding therapy period may be the particular therapy period associated with block 906. In some embodiments, the glucose level control system 300 receives the measured time-varying glucose level data from the glucose level sensor 310 or from one or more isolated glucose measurements 406. Alternatively, or in addition, the measured glucose level may include a plurality of measured glucose levels. The plurality of measured glucose levels may correspond to a single medicament dose or multiple medicament doses. For example, the plurality of measured glucose levels may correspond to the one or more medicament doses administered at block 906. In some embodiments, the block 910 may include one or more of the embodiments previously described with respect to the blocks 706, 708, and/or 808.

At block 912, the glucose level control system 300 may determine a difference between the predicted glucose level and the measured glucose level. Alternatively, or in addition, the glucose level control system 300 determines the difference between a plurality of predicted glucose levels and a plurality of measured glucose levels. For example, for each of a set of time periods or points in time, the glucose level control system 300 may determine a difference between a measured glucose level value and a corresponding predicted glucose level value associated with the time period.

In some embodiments, the glucose level control system 300 determines the difference between the predicted and measured glucose levels by determining an average difference between the plurality of predicted glucose level values and the plurality of measured glucose level values. Alternatively, or in addition, the glucose level control system 300 may determine the difference between the predicted and measured glucose levels by determining a trend in the difference of the plurality of predicted glucose level values and the plurality of measured glucose level values. In some embodiments, the block 912 may include one or more of the embodiments previously described with respect to the block 710.

At block 914, responsive to determining that the difference between the measured glucose level and the predicted glucose level satisfies a threshold difference, the glucose level control system 300 adapts the prediction model. Alternatively, or in addition, the glucose level control system 300 may adapt the prediction model in response to the difference between a plurality of measured and predicted glucose level values satisfying a threshold difference. The threshold difference may be ±1 mg/dL, 10 mg/dL, 15 mg/dL, 20 mg/dL, 40 mg/dL, more than 40 mg/dL, or any number in between. In some embodiments, adapting the prediction model causes a change in the medicament doses determined by the control algorithm. For example, the change in the prediction model may correspond to a change in size of the one or more medicament doses and/or a change in the timing of administering of the one or more medicament doses. Alternatively, or in addition, adapting the prediction model may cause a change in the predicted glucose level determined by the control algorithm.

In some embodiments, the glucose level control system 300 adapts the prediction model if it determines that a difference between a minimum number or percentage of the plurality of measured glucose levels and corresponding plurality of predicted glucose levels satisfies the threshold difference. The minimum number of differences that satisfy the threshold difference may vary from 1, 10, 50, 100, 1,000, 10,000, or more than 10,000, or any number in between. Similarly, the minimum percentage of differences that satisfy the threshold difference may vary from 1%, 10%, 30%, 50%, 100%, or any percentage in between. In some embodiments, the glucose level control system 300 may adapt the prediction model if it determines that an average difference between the plurality of measured glucose levels and the corresponding plurality of predicted glucose levels satisfies the threshold difference. Alternatively, or in addition, the glucose level control system 300 may adapt the prediction model if it determines that a trend in a difference between the plurality of measured glucose levels and the corresponding plurality of predicted glucose levels satisfies the threshold difference.

In some embodiments, the glucose level control system 300 adapts the prediction model by modifying the weight of one or more parameters. It should be understood that increasing the weight of a parameter may increase the importance of the parameter to the prediction model and decreasing the weight of a parameter may decrease the importance of a parameter to the prediction model. In some embodiments, the glucose level control system 300 may adapt the prediction model by modifying the weight of a glucose level velocity and/or glucose level acceleration parameter. The glucose velocity parameter may correspond to the speed at which the subjects 512 glucose level changes, and the glucose level acceleration parameter may correspond to a change in the speed at which the glucose level of the subject 512 changes.

Alternatively, or in addition, the glucose level control system 300 may adapt the prediction model by modifying the relationship between the medicament doses determined by the control algorithm and the predicted glucose levels. For example, the prediction model may be adapted to respond more aggressively to predicted glucose levels that are high or low (e.g., predicted glucose levels that are near or past the limits of a target range). For instance, if the predicted glucose levels are high, the glucose level control system 300 may determine larger medicament doses compared to the medicament doses determined by a prediction model that has not been adapted to be more aggressive. Alternatively, or in addition, the prediction model may be adapted to respond less aggressively to predicted glucose levels which are high or low.

In some embodiments, the glucose level control system 300 may adapt the prediction model by modifying the relationship between the medicament on board value and the predicted glucose levels. The glucose level control system 300 may adapt the prediction model so that the medicament on board value has more or less weight in the control algorithm used to predict glucose levels. Alternatively, or in addition, the glucose level control system 300 may adapt the prediction model so that changes in the medicament on board value cause larger changes in the predicted glucose level, as compared to a prediction model that has not been adapted. In some embodiments, the glucose level control system 300 may adapt the prediction model so that changes in the medicament on board value cause smaller changes in the predicted glucose level, as compared to a prediction model that has not been adapted.

In some embodiments, the glucose level control system 300 may adapt the prediction model by modifying the relationship between one or more characteristics of the subject 512 and the predicted glucose levels. The characteristics of the subject 512 may include the weight, total daily dose of a medicament, age, puberty status, gender, and/or sex of the subject 512. The glucose level control system 300 may adapt the prediction model so that one or more of the characteristics of the subject 512 have more or less weight in the control algorithm used to predict glucose levels. In some embodiments, the glucose level control system 300 may adapt the prediction model so that changes in one or more characteristics of the subject 512 causes larger changes in the predicted glucose level, as compared to changes made by a prediction model that has not been adapted. Alternatively, or in addition, the glucose level control system 300 may adapt the prediction model so that changes in one or more characteristics of the subject 512 cause smaller changes in the predicted glucose level, as compared to changes made by a prediction model that has not been adapted.

In some embodiments, the glucose level control system 300 may adapt the prediction model by modifying a selection of historical data used by the prediction model to determine the predicted glucose levels of the subject 512. For example, the glucose level control system 300 may change one or more evaluation criteria that are used to determine what historical data is used by the prediction model. Alternatively, or in addition, the glucose level control system 300 may adapt the prediction model by changing the range of the historical data that is used. For example, the glucose level control system 300 may expand the range of the historical data from one day to three days.

In some embodiments, the glucose level control system 300 may adapt the prediction model by changing a value of one or more parameters of the prediction model. For example, the glucose level control system 300 may adapt the prediction model by changing a value corresponding to the target setpoint parameter, the subject model parameter a glucose level prediction parameter, dosing aggressiveness parameter, dosing setting parameter, the insulin pharmacokinetic parameter, the insulin sensitivity parameter, and/or any other parameter used by the prediction model. In some embodiments, values corresponding to certain parameters may be excluded from the adaptation process. For example, the glucose level control system 300 may prevent the value of parameters corresponding to the weight, age, ethnicity, and/or race of the subject 512 from being changed.

Alternatively, or in addition, the glucose level control system 300 may adapt the prediction model by modifying one or more relationships between two or more parameters of the prediction model. For example, if a first parameter is based at least in part on a second parameter, the glucose level control system 300 may adapt the prediction model by increasing the weight of the second parameter in relation to the other parameters used to determine the first parameter.

In some embodiments, the glucose level control system 300 may perform the process 900 to adapt the prediction model to improve the accuracy of the glucose level prediction. For example, adapting the prediction model may reduce the error between the predicted glucose levels and the corresponding measured glucose levels. The glucose level control system 300 may use the adapted prediction model to provide medicament doses that better manage the subject's disease. For example, the medicament doses based on the adapted model may include reducing the fluctuations of the subject's 512 glucose level and/or increasing the amount of time the subject's 512 glucose level stays within a desired range.

### Total Daily Dose Adaptation Process

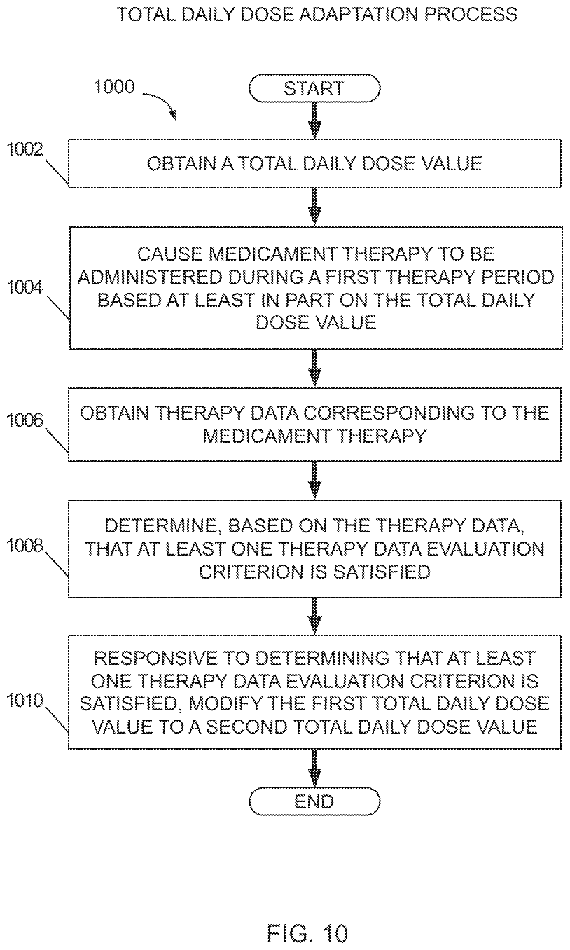

FIG. 10 presents a flowchart of an example total daily dose adaptation process 1000 in accordance with certain embodiments. The process 1000 may be performed by any system that can adapt a total daily dose value corresponding to a total daily dose of medicament administered to the subject 512. For example, the process 1000 may be performed by an ambulatory medicament device 250, the glucose level control system 300, and/or an electronic device 252. In some embodiments, the process 1000 may be at least partly performed by one or more elements of the glucose level control system 300, such as one or more processors 530, one or more controllers 202, the memory 540, the medicament delivery interface 306, or the interface circuitry 532. In some cases, at least certain operations of the process 1000 may be performed by the remote computing system 254 that receives therapy data corresponding to the subject 512. Although one or more different systems may perform one or more operations of the process 1000, to simplify discussions and not to limit the present disclosure, the process 1000 is described with respect to particular systems.

The glucose level control system 300 may perform the process 1000 for the same events, circumstances, and time-intervals as the process 700 and the process 800. For example, the process 1000 may be performed if the subject 512 is switching to a new medicament type, if the subject's 512 medicament administration schedule has been modified, if the subject has recently started using the glucose level control system 300, etc.

The process 1000 begins at block 1002 where, for example, the glucose level control system 300 obtains a total daily dose value. The glucose level control system 300 may receive the total daily dose value via user interaction with a user interface. For example, the user interface may prompt the user to enter one or more total daily dose values corresponding to the subject 512. Alternatively, or in addition, the glucose level control system 300 may receive the total daily dose value from the electronic device 252 or the remote computing system 254. The total daily dose value may be based at least in part on an estimated total daily dose of medicament administered to the subject 512. Alternatively, or in addition, the total daily dose value may be based at least in part on an average total daily dose of medicament administered to the subject 512. Furthermore, the total daily dose value may be based at least in part on therapy data corresponding to one or more past therapy periods. In some embodiments, the total daily dose value may correspond to a particular therapy period. The therapy period may be a period between two events. Alternatively, or in addition, the therapy period may be a time period of minutes, hours, or days. In some cases, the time period is at least a day. In some embodiments, the block 1002 may include one or more of the embodiments previously described with respect to the blocks 702 and/or 802.

At block 1004, the glucose level control system 300 causes a medicament therapy to be administered to a subject. In some embodiments, the medicament therapy may include one or more basal doses and/or non-basal doses (e.g., food-intake dose, correction dose, etc.). The medicament therapy may be based at least in part on the total daily dose value obtained at block 1002. For example, the size and frequency of a basal dose may be based at least in part on the total daily dose value. Alternatively, or in addition, the basal dose may be determined based at least in part on a basal rate. Furthermore, the basal rate may be determined based at least in part on the total daily dose value. In some embodiments, the size of one or more correction doses corresponding to the medicament therapy may be based at least in part on the total daily dose value. Similarly, the size of one or more food-intake doses corresponding to the medicament therapy may be based at least in part on the total daily dose value.

Alternatively, or in addition, the medicament therapy may be based at least in part on the glucose level of the subject 512. In some embodiments, the glucose level control system 300 receives the glucose level of the subject 512 from the glucose level sensor 310 or from one or more isolated glucose measurements 406. The glucose level control system 300 may determine the medicament therapy using one or more controllers 202 and/or a control algorithm, which may generate one or more dose control signal based at least in part on the total daily dose value received at the block 1002. Alternatively, or in addition, the dose control signal may be based at least in part on predicted glucose level data. In some embodiments, the predicted glucose level data may be based at least in part on the total daily dose value, a medicament on board value, and/or one or more characteristics of the subject 512 (e.g., weight, age, puberty status, gender, etc.). Alternatively, or in addition, the predicted glucose level data may be determined using a prediction model.

The medicament delivery device 312 may administer the medicament therapy in response to the dose control signal. In some embodiments, the medicament therapy may be delivered during a particular therapy period. For example, the medicament therapy may be delivered during the therapy period associated with the total daily dose value obtained at block 1002. In some embodiments, the block 1004 may include one or more of the embodiments previously described with respect to the blocks 704, 706, 804, 902, 908, 910, and/or 914.

At block 1006, the glucose level control system 300 obtains therapy data corresponding to the medicament therapy administered during a particular therapy period. In some embodiments, the therapy data corresponds to the therapy period associated with the total daily dose value obtained at block 1002. Alternatively, or in addition, the therapy data may correspond to one or more past therapy periods. As described above, the therapy data may correspond to past or future (e.g., scheduled) medicament deliveries. For example, the therapy data may include the size, type, timing, and/or quantity of medicament doses. Alternatively, or in addition, the therapy data may include glucose level data corresponding to the subject 512. In some embodiments, the block 1006 may include one or more of the embodiments previously described with respect to the blocks 702, 802, and/or 1002.

At block 1008, the glucose level control system 300 determines, based at least in part on the therapy data, that at least one of a plurality of therapy data evaluation criteria is satisfied. In some embodiments, a therapy data evaluation criterion may be satisfied if the glucose level control system 300 determines that a total daily dose value does not match an administered daily medicament dose value within a threshold degree. The total daily dose value may be the total daily dose value obtained at block 1002. Alternatively, or in addition, the administered daily medicament dose value may correspond to the medicament administered during the therapy period associated with the total daily dose value obtained at block 1002.

In some embodiments, the threshold degree may be a percent difference. In some cases, the threshold degree may be a percent difference of 1%, 5%, 10%, 30%, 60%, or more than 60%, or any percentage in between. For instance, the therapy data evaluation criterion may be satisfied if the percent difference between the total daily dose value and the administered daily medicament dose value is greater than 20%. Alternatively, or in addition, the threshold degree may be an absolute difference. In some embodiments, the threshold degree is an absolute difference of 1 unit of medicament, 10 units of medicament, 15 units of medicament, 20 units of medicament, 40 units of medicament, more than 40 units of medicament, or any number in between. For example, the therapy data evaluation criterion may be satisfied if the absolute difference between the total daily dose value and the administered daily medicament dose value is greater than 30 units of insulin.

In some embodiments, a therapy data evaluation criterion may be satisfied if the glucose level control system 300 determines that the difference between the total daily dose value and the administered daily medicament dose value exceeds a difference threshold for a minimum number of days during one or more therapy periods. As described above, the difference threshold may be a difference percentage or an absolute difference. In some embodiments, the minimum number of days may vary from 1 day, 2 days, 7 days, 14 days, or more than 14 days, or any number of days in between. For example, a therapy data evaluation criterion may be satisfied if the difference between the total daily dose value and the administered daily medicament dose value exceeds 30 units of insulin for at least three days during the corresponding therapy period. In some embodiments, the days must be consecutive days. Alternatively, or in addition, the minimum number of days may be non-consecutive days.

In some embodiments, a therapy data evaluation criterion may be satisfied if the glucose level control system 300 determines that a trend of the difference between the total daily dose value and the administered daily medicament dose value exceeds a trend threshold. In some embodiments, the trend threshold may be a percent increase of 1%, 5%, 10%, 30%, 60%, or more than 60%, or any percentage in between, during a certain time period. The time period over which the rate of change is evaluated may also vary. For example, the time period may be 30 minutes, 1 hour, 12 hours, 1 day, 1 week, or more than 1 week, or any time period in between. For example, the therapy data evaluation criterion may be satisfied if the difference between the total daily dose value and the administered daily medicament dose value is increasing by more than 10% per day. In some embodiments, the trend threshold may be a rate of change that is greater than 1 unit of medicament, 2 units of medicament, 5 units of medicament, 10 units of medicament, 40 units of medicament, or more than 40 units of medicament, or any amount in between, during a certain time period. For example, the therapy data evaluation criterion may be satisfied if the difference between the total daily dose value and the administered daily medicament dose value is increasing by more than 4 units of medicament per day.

In some embodiments, a therapy data evaluation criterion may be satisfied if the glucose level control system 300 determines that the average difference between the total daily dose value and the administered daily medicament dose value during one or more therapy periods exceeds an average difference threshold. Like the threshold degree describe above, the average difference threshold may vary and can be a percentage or an absolute amount. For example, the therapy data evaluation criterion may be satisfied if the average difference between the total daily dose value and the administered daily medicament dose value is greater than 5 units of insulin. In order to determine an average difference, the glucose level control system 300 may divide a therapy period into intervals. For example, a therapy period may be divided into days, and the average may be the average daily difference. Alternatively, or in addition, the therapy period may be divided into minutes, hours, or weeks.

In some embodiments, a therapy data evaluation criterion may be satisfied if the glucose level control system 300 determines that a threshold amount of time has passed since the total daily dose value was adapted. In some embodiments, the threshold amount of time may be 30 minutes, 1 hour, 12 hours, 1 day, 1 week, or more than 1 week, or any time period in between. Alternatively, or in addition, a therapy data evaluation criterion may be satisfied if the glucose level control system 300 determines that a one or more therapy periods have passed since the total daily dose value was adapted.

In some embodiments, a therapy data evaluation criterion may be satisfied if the glucose level control system 300 determines a particular time period has been reached. The particular time period may correspond to a particular time of day. For example, the particular time period may correspond to a morning, afternoon, evening, or night time period. Alternatively, or in addition, the particular time period may correspond to a particular day of the week. For example, the therapy data evaluation criterion may be satisfied if the glucose level control system 300 determines that Wednesday has been reached.

At block 1010, responsive to determining that at least one of the plurality of therapy data evaluation criteria is satisfied, the glucose level control system 300 modifies the total daily dose value from a first total daily dose value to a second total daily dose value. In some embodiments, the first total daily dose is the total daily dose value obtained at block 802, and corresponds to a first therapy period (e.g., the therapy period associated with the total daily dose value obtained at block 802). The second total daily dose value may correspond to a second therapy period. In some embodiments, the second total daily dose value is based at least in part on the therapy data, a medicament on board value, and/or the glucose levels of the subject 512. Furthermore, the glucose level control system 300 may use the control algorithm to generate a dose control signal based at least in part on the second total daily dose value.

In some embodiments, adapting the total daily dose value may change the medicament therapy that the glucose level control system 300 causes to be administered to the subject 512. For example, since the dose control signal may be based at least in part on the total daily dose value, adapting the total daily dose to the second total daily dose value may change the dose control signal. For example, adapting the total daily dose value may modify the size of the basal dose or a basal rate used to determine the basal dose corresponding to the medicament therapy that corresponds to the second therapy period. Alternatively, or in addition, adapting the total daily dose value may modify the size of one or more food-intake doses and/or correction doses corresponding to the medicament therapy that corresponds to the second therapy period.

In some embodiments, the glucose level control system 300 may perform the process 1000 to adapt the total daily dose value to reduce the error between the total daily dose value obtained at block 1002 and the daily medicament dose that was administered to the subject 512 during the corresponding therapy period. The glucose level control system 300 may use the adapted total daily dose value to provide medicament doses that better manage the subject's disease. For example, the medicament doses based on the adapted total daily dose value may reduce the fluctuations of the subject's 512 glucose level and/or increase the amount of time the subject's 512 glucose level stays within a desired range. It should be understood that the glucose level control system 300 may repeat the process 1000 multiple times to further adapt the total daily dose value. For example, the glucose level control system 300 may perform the process 1000 a second time and may modify the second total daily dose value to a third total daily dose value.

### EXAMPLE EMBODIMENTS

Some example enumerated embodiments are recited in this section in the form of methods, systems, and non-transitory computer-readable media, without limitation.

In other embodiments, a system or systems may operate according to one or more of the methods and/or computer-readable media recited in the preceding paragraphs. In yet other embodiments, a method or methods may operate according to one or more of the systems and/or computer-readable media recited in the preceding paragraphs. In yet more embodiments, a computer-readable medium or media, excluding transitory propagating signals, may cause one or more computing devices having one or more processors and non-transitory computer-readable memory to operate according to one or more of the systems and/or methods recited in the preceding paragraphs.

### TERMINOLOGY

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein. Further, it should be understood that certain embodiments may be combinable or combined with certain other embodiments described herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware. Further, the computing system may include, be implemented as part of, or communicate with a glucose level control system, an ambulatory medicament system, or an ambulatory medical device.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. A computing environment can include any type of computer system, including, but not limited to, a computer system based on a microprocessor, a mainframe computer, a digital signal processor, a portable computing device, a device controller, or a computational engine within an appliance, to name a few.

Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Disjunctive language such as the phrase “at least one of X, Y, or Z,” unless specifically stated otherwise, is otherwise understood with the context as used in general to present that an item, term, etc., may be either X, Y, or Z, or any combination thereof (for example, X, Y, and/or Z). Thus, such disjunctive language is not generally intended to, and should not, imply that certain embodiments require at least one of X, at least one of Y, or at least one of Z to each be present.

Unless the context clearly requires otherwise, throughout the description and the claims, the words “comprise,” “comprising,” and the like are to be construed in an inclusive sense, as opposed to an exclusive or exhaustive sense, i.e., in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words using the singular or plural number may also include the plural or singular number respectively. The word “or” in reference to a list of two or more items, covers all of the following interpretations of the word: any one of the items in the list, all of the items in the list, and any combination of the items in the list. Likewise the term “and/or” in reference to a list of two or more items, covers all of the following interpretations of the word: any one of the items in the list, all of the items in the list, and any combination of the items in the list.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

It should be emphasized that many variations and modifications may be made to the above-described embodiments, the elements of which are to be understood as being among other acceptable examples. All such modifications and variations are intended to be included herein within the scope of this disclosure.

What is claimed is:

1. A glucose level control system configured to adapt a basal dose of medicament administered to a subject over a first time period, the glucose level control system comprising:
a medicament delivery interface configured to operatively connect to a medicament pump for infusing the medicament into the subject;
a memory configured to store specific computer-executable instructions; and
a hardware processor in communication with the memory and configured to execute the specific computer-executable instructions to at least:
obtain, via user interaction with a basal dose entry user interface, an indication of a first basal dose associated with administering the medicament to the subject over the first time period, wherein the first time period is on the order of at least a day;
configure the medicament delivery interface to administer the medicament using at least in part the first basal dose over the first time period;
determine a predicted glucose level of the subject over the first time period based at least in part on a medicament on board value over the first time period;
determine a measured glucose level of the subject during the first time period;
determine a difference between the predicted glucose level of the subject and the measured glucose level; and
adapt the basal dose of the medicament by modifying the first basal dose to a second basal dose associated with administering the medicament to the subject over a second time period, wherein the second basal dose is based at least in part on the difference between the predicted glucose level of the subject and the measured glucose level.

2. The glucose level control system of claim 1, further comprising a glucose level sensor interface configured to receive a glucose level signal from a glucose level sensor operatively connected to the subject, wherein the measured glucose level of the subject is determined based at least in part on the glucose level signal.

3. The glucose level control system of claim 1, wherein the second basal dose is selected automatically without user interaction with the basal dose entry user interface.

4. The glucose level control system of claim 1, wherein the first basal dose comprises a first basal rate and the second basal dose comprises a second basal rate.

5. The glucose level control system of claim 1, wherein the first basal dose comprises a single medicament dose.

6. The glucose level control system of claim 1, wherein the first basal dose comprises a long-acting insulin dose.

7. The glucose level control system of claim 1, wherein the first time period comprises a plurality of time segments.

8. The glucose level control system of claim 7, wherein the hardware processor is further configured to execute the specific computer-executable instructions to at least select a time segment from the plurality of time segments where at least one of a plurality of basal evaluation criteria is satisfied, wherein the plurality of basal evaluation criteria comprises:
a medicament on board attributable to non-basal medicament delivery is less than or equal to a medicament delivery threshold;
a medicament bolus beyond a minimum bolus threshold to compensate for food intake occurred at least 3 hours prior to a beginning of the time segment;
the medicament on board attributable to a food-intake bolus is less than or equal to 2 units;
a medicament bolus beyond the minimum bolus threshold to compensate for a glucose excursion occurred at least 3 hours prior to the beginning of the time segment;
the medicament on board attributable to a correction bolus is less than or equal to 2 units;
a glucose level of the subject is within a target range;
it is determined that the subject is not exercising;
it is determined that the subject is not experiencing a temporary illness that affects the glucose level of the subject; and
the medicament on board attributable to food intake and to the correction bolus is less than or equal to 3 units.

9. The glucose level control system of claim 8, wherein the measured glucose level corresponds to the time segment where the at least one of the plurality of basal evaluation criteria is satisfied.

10. The glucose level control system of claim 1, wherein the first time period comprises a receding time horizon.

11. The glucose level control system of claim 1, wherein the medicament on board value is based at least in part on one or more non-basal doses.

12. The glucose level control system of claim 11, wherein the one or more non-basal doses comprise at least one of a food-intake dose or a correction bolus.

13. The glucose level control system of claim 11, wherein the medicament on board value is further based at least in part on the first basal dose.

14. The glucose level control system of claim 1, wherein the predicted glucose level is based at least in part on a food-intake dose or a correction bolus, and the measured glucose level corresponds to the food-intake dose or the correction bolus.

15. The glucose level control system of claim 1, wherein the hardware processor is further configured to execute the specific computer-executable instructions to at least obtain a total daily dose of the medicament for the subject, and wherein the second basal dose is based at least in part on the total daily dose of the medicament.

16. The glucose level control system of claim 1, wherein the hardware processor is further configured to execute the specific computer-executable instructions to at least obtain a body weight of the subject, and wherein the second basal dose is based at least in part on the body weight of the subject.

17. The glucose level control system of claim 1, wherein the predicted glucose level comprises a plurality of predicted glucose level values.

18. The glucose level control system of claim 17, wherein one or more of the plurality of predicted glucose level values correspond to different times during the first time period.

19. The glucose level control system of claim 17, wherein the plurality of predicted glucose level values is associated with different medicament on board values corresponding to different occurrences of medicament doses that have been administered to the subject.

20. The glucose level control system of claim 17, wherein the measured glucose level comprises a plurality of measured glucose level values.

21. The glucose level control system of claim 20, wherein the plurality of measured glucose level values is associated with a single medicament dose and wherein one or more of the plurality of measured glucose level values correspond to different times during the first time period.

22. The glucose level control system of claim 20, wherein the plurality of measured glucose level values is associated with different doses administered to the subject, and wherein the plurality of measured glucose level values corresponds to the first time period.

23. The glucose level control system of claim 20, wherein determining the difference between the predicted glucose level and the measured glucose level comprises determining the difference between the plurality of predicted glucose level values and the plurality of measured glucose level values.

24. The glucose level control system of claim 23, wherein determining the difference between the predicted glucose level and the measured glucose level comprises determining an average difference between the plurality of predicted glucose level values and the plurality of measured glucose level values.

25. The glucose level control system of claim 23, wherein determining the difference between the predicted glucose level and the measured glucose level comprises determining a trend in the difference of the plurality of predicted glucose level values and the plurality of measured glucose level values.

26. The glucose level control system of claim 23, wherein determining the difference between the predicted glucose level and the measured glucose level comprises determining a maximum difference between the plurality of predicted glucose level values and the plurality of measured glucose level values.

27. The glucose level control system of claim 20, wherein the second basal dose is based at least in part on the difference between the plurality of predicted glucose level values and the plurality of measured glucose level values.

28. A computer-implemented method of adapting a basal dose of medicament administered to a subject over a first time period, the computer-implemented method comprising:
by a hardware processor of a glucose level control system configured to adapt the basal dose of the medicament,
obtaining, via user interaction with a basal dose entry user interface, an indication of a first basal dose associated with administering the medicament to the subject over the first time period, wherein the first time period is on the order of at least a day;
causing the medicament to be administered based at least in part on the first basal dose over the first time period;
determining a predicted glucose level of the subject over the first time period based at least in part on a medicament on board value over the first time period;
determining a measured glucose level of the subject during the first time period;
determining a difference between the predicted glucose level of the subject and the measured glucose level; and
adapting the basal dose of the medicament by modifying the first basal dose to a second basal dose associated with administering the medicament to the subject over a second time period, wherein the second basal dose is based at least in part on the difference between the predicted glucose level of the subject and the measured glucose level.

29. The computer-implemented method of claim 28, further comprising receiving a glucose level signal from a glucose level sensor operatively connected to the subject, wherein the measured glucose level of the subject is determined based at least in part on the glucose level signal.

30. The computer-implemented method of claim 28, wherein the second basal dose is selected automatically without user interaction with the basal dose entry user interface.

31. The computer-implemented method of claim 28, wherein the first basal dose comprises a first basal rate and the second basal dose comprises a second basal rate.

32. The computer-implemented method of claim 28, wherein the first basal dose comprises a single medicament dose.

33. The computer-implemented method of claim 28, wherein the first basal dose comprises a long-acting insulin dose.

34. The computer-implemented method of claim 28, wherein the first time period comprises a plurality of time segments.

35. The computer-implemented method of claim 34, further comprising selecting a time segment from the plurality of time segments where at least one of a plurality of basal evaluation criteria is satisfied, wherein the plurality of basal evaluation criteria comprises:
a medicament on board attributable to non-basal medicament delivery is less than or equal to a medicament delivery threshold;
a medicament bolus beyond a minimum bolus threshold to compensate for food intake occurred at least 3 hours prior to a beginning of the time segment;
the medicament on board attributable to a food-intake bolus is less than or equal to 2 units;
a medicament bolus beyond the minimum bolus threshold to compensate for a glucose excursion occurred at least 3 hours prior to the beginning of the time segment;
the medicament on board attributable to a correction bolus is less than or equal to 2 units;
a glucose level of the subject is within a target range;
it is determined that the subject is not exercising;
it is determined that the subject is not experiencing a temporary illness that affects the glucose level of the subject; and
the medicament on board attributable to food intake and to the correction bolus is less than or equal to 3 units.

36. The computer-implemented method of claim 35, wherein the measured glucose level corresponds to the time segment where the at least one of the plurality of basal evaluation criteria is satisfied.

37. The computer-implemented method of claim 28, wherein the first time period comprises a receding time horizon.

38. The computer-implemented method of claim 28, wherein the medicament on board value is based at least in part on one or more non-basal doses.
